(19) United States
(12) Patent Application Publication
(10) Pub. No.: US 2024/0181444 A1
(43) Pub. Date: Jun. 6, 2024

(54) MULTIPLEX DEVICES AND METHODS FOR PATHOGEN DETECTION

(71) Applicant: University of Florida Research Foundation, Inc., Gainesville, FL (US)

(72) Inventors: Zhonghui Hugh Fan, Gainesville, FL (US); Carlos Manzanas, Gainesville, FL (US); Morteza Alipanahrostami, Gainesville, FL (US); John A. Lednicky, Gainesville, FL (US); Chang-Yu Wu, Gainesville, FL (US)

(21) Appl. No.: 18/577,356

(22) PCT Filed: Sep. 15, 2022
(86) PCT No.: PCT/US2022/076456

§ 371 Date: Jan. 8, 2024

Related U.S. Application Data
(60) Provisional application No. 63/261,373, filed on Sep. 20, 2021.

(51) Int. Cl. B01L 3/00; C12Q 1/6844

(52) U.S. Cl. CPC B01L 3/5025; C12Q 1/6844; B01L 2200/16; B01L 2300/0829; B01L 2300/1827; B01L 2400/0616

(57) ABSTRACT

Provided are multiplex devices for nucleic acid detection and methods for nucleic acid detection. The device includes at least two parallel buffer units comprised of buffer wells arranged in a row. The buffer units are connected to form a multiplex device. A mixing unit is slides along a bottom of each buffer unit to align with each buffer well in turn. When the mixing unit is aligned with a buffer well, a ball valve releases buffer from the buffer well into the mixing well. The device includes a detection unit removably coupled to the bottom of each mixing unit to receive fluids from the mixing well.

### CROSS-REFERENCE TO RELATED APPLICATIONS

This application claims the benefit of and priority to U.S. Provisional Application Ser. No. 63/261,373, having the title “MULTIPLEX DEVICES AND METHODS FOR PATHOGEN DETECTION”, filed on Sep. 20, 2021, the disclosure of which is incorporated herein by reference in its entirety.

### STATEMENT REGARDING FEDERALLY SPONSORED RESEARCH OR DEVELOPMENT

This invention was made with government support under Grant Numbers R01AI158868 and R01AI155735, awarded by the National Institute of Health; and Grant Number 2030844, awarded by the National Science Foundation. The government has certain rights in the invention.

### BACKGROUND

Early and accurate detection of severe acute respiratory syndrome coronavirus 2 (SARS-CoV-2) and influenza viruses at the point-of-care (POC) is needed for reducing disease transmission during the current pandemic and future flu seasons. Severe acute respiratory syndrome coronavirus 2 (SARS-CoV-2) is the causative agent of the ongoing pandemic of coronavirus disease 2019 (COVID-19), which has resulted in over 4.3 million deaths worldwide as of Aug. 10, 2021. The main challenges associated with the COVID-19 pandemic include non-specific clinical symptoms such as fever, a large number of asymptomatic individuals who are undiagnosed and contribute to the disease transmission, and lack of rapid diagnostic tools. With expected co-circulation of respiratory viruses such as influenza viruses during flu seasons, these challenges require early and rapid virus detection at the point-of-care (POC) for reducing disease transmission. SARS-CoV-2 and influenza viruses can cause contagious respiratory illnesses with similar symptoms; thus, it is important to have an ability to tell them apart by detecting these two viruses simultaneously for clinical and resource management.

### SUMMARY

Embodiments of the present disclosure provide multiplex devices for detecting nucleic acid from a pathogen, methods for detecting nucleic acids from a pathogen using multiplex devices, and the like.

An embodiment of the present disclosure includes a multiplex device for preparing a sample for nucleic acid detection. The device includes at least two parallel buffer units comprising a plurality of buffer wells arranged in a row, wherein each buffer well comprises a ball valve in a bottom of the well. A mixing unit is associated with each buffer unit, the mixing unit having a mixing well and a pin. The mixing unit is slidably connected to a bottom of the buffer unit such that the mixing unit can be moved along a length of the buffer unit to align with each buffer well in turn. When the mixing unit is aligned with a buffer well, the pin engages with the ball valve to release buffer from the buffer well into the mixing well. The device includes a detection unit removably coupled to the bottom of each mixing unit to receive fluids from the mixing well. The parallel buffer units are connected to form a multiplex device.

An embodiment of the present disclosure also includes a multiplex device for detecting nucleic acid from a nucleic acid source. The device includes at least two parallel buffer units, wherein each buffer unit comprises a plurality of buffer wells arranged in a row, wherein each buffer well comprises a ball valve in a bottom of the well. A nucleic acid source and a first buffer is provided to a first buffer well, a second buffer is provided to a second buffer well, a third buffer is provided to a third buffer well, and a fourth buffer is provided to a fourth buffer well. An amplification unit is coupled to an end of each buffer unit. The amplification unit includes a buffer well such that the coupling forms a singleplex unit. If the nucleic acid source is DNA the amplification unit is a LAMP unit and if the nucleic acid source is RNA the amplification unit is an RT-LAMP unit. An amplification buffer is provided to an amplification buffer well. A mixing unit is associated with each singleplex unit. The mixing unit comprises a mixing well and a pin. The mixing unit is slidably connected to a bottom of the singleplex unit such that the mixing unit can be moved along a length of the singleplex unit to align with each buffer well in turn, beginning with the first buffer well and ending with the amplification unit. When the mixing unit is aligned with a buffer well, the pin engages with the ball valve to release buffer from the buffer well into the mixing well such that nucleic acid is produced in the mixing unit from the nucleic acid source after the mixing unit engages with the amplification unit. A detection unit is removably coupled to the bottom of each mixing unit to receive buffer and nucleic acid from the mixing well, wherein the nucleic acid is collected and amplified in the detection unit. A drain unit is removably coupled to the bottom of each detection unit to extract buffer from the detection unit. The device also includes a heating unit to amplify the nucleic acid product. The heating unit comprises a seat for the detection unit, wherein the heating unit provides a temperature of 57° C. to 67° C.

An embodiment of the present disclosure also includes a method for preparing a sample to be used for detecting nucleic acid from a nucleic acid source. The method includes placing a first liquid sample in at least a first well of a first buffer unit of a nucleic acid detecting apparatus. Then at least a second, third and fourth liquid are placed in at least second, third and fourth wells, respectively, of the first buffer unit of the nucleic acid detecting apparatus. A second liquid sample is placed in at least a first well of a second buffer unit of the nucleic acid detecting apparatus. At least a second, third and fourth liquid are placed in at least second, third and fourth wells, respectively, of the second buffer unit of the nucleic acid detecting apparatus. A mixing unit is slidably coupled to a bottom of each buffer unit allowing a user to create relative movement between the mixing unit and the buffer unit by exerting a manual force on at least one of the buffer unit and the mixing unit. The method includes exerting the manual force on at least one of the buffer unit and the mixing unit to slide the mixing unit along the buffer unit to cause the first, second, third and fourth wells to come into temporary alignment with the mixing unit for respective time periods. A valve mechanism in each of the wells causes the first, second, third and fourth liquids to be released from the first, second, third and fourth wells, respectively, into the mixing unit, in turn, when the manual force is exerted to cause the second, third and fourth wells to come into temporary alignment with the mixing unit for respective time periods such that each liquid sample is prepared for nucleic acid amplification as it combines with the buffers from the wells in the mixing unit. A detection unit is coupled to a bottom of the mixing unit and receives fluid from the mixing unit.

Other compositions, apparatus, methods, features, and advantages will be or become apparent to one with skill in the art upon examination of the following drawings and detailed description. It is intended that all such additional compositions, apparatus, methods, features and advantages be included within this description, be within the scope of the present disclosure, and be protected by the accompanying claims.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIGS. 1.1A-1.1B show a perspective view of an exploded (1.1A) and (1.1B) assembled VLEAD in accordance with embodiments of the present disclosure.

FIGS. 1.2A-1.2C show pin in slot mechanism components, (1.2A) the slot, (1.2B) the pin, (1.2C) cross-sectional view of assembled mechanism in accordance with embodiments of the present disclosure.

FIGS. 1.3A-1.3B show: Exploded (1.3A) and assembled (1.3B) views of the VLEAD in accordance with embodiments of the present disclosure.

FIGS. 1.4A-1.4B show: Exploded (1.4A) and assembled (1.4B) views of the VLEAD in accordance with embodiments of the present disclosure.

FIG. 1.5 is a camera image of an example of real-time LAMP-enabled diagnostic platform in accordance with embodiments of the present disclosure.

FIG. 2.1 provides an exploded view of the 2-plex VLEAD in accordance with embodiments of the present disclosure.

FIG. 2.2 provides an exploded view of the 3-plex VLEAD in accordance with embodiments of the present disclosure.

FIG. 2.3 provides an exploded view of a single detection unit in accordance with embodiments of the present disclosure.

FIG. 2.4 provides an exploded view of the 4-plex VLEAD in accordance with embodiments of the present disclosure.

FIG. 2.5 provides an exploded view of the VLEAD for integration with aerosol collection in accordance with embodiments of the present disclosure.

FIG. 3.1A provides an exploded view of a 2-plex VLEAD in accordance with embodiments of the present disclosure. In each well of the buffer unit one stainless steel ball is placed at the bottom that functions as the valve as explained in FIG. 3.1B. FIG. 3.1C is a photograph of an assembled device, in which the mixing unit slides to the second well from the left to lift the ball in the well. FIG. 3.1D is a top view picture of a detection unit, showing a well in a polycarbonate sheet. FIG. 3.1E is a bottom view of a detection unit, showing the paper pad underneath the well layer.

FIG. 3.2A shows real-time RT-LAMP amplification for SARS-CoV-2 showing fluorescent signal of 10⁴, 10³, 10², and 10 genome equivalents (GEs) as a function of reaction time. FIG. 3.2B shows the calibration curve between the threshold time (Ct) and SARS-CoV-2 GEs in each reaction (in log scale). FIG. 3.2C shows the real-time RT-LAMP amplification for influenza A virus, showing fluorescent signal of 600, 60, 6, and 1.2 Median Tissue Culture Infectious Dose (TCID₅₀) of influenza A virus as a function of RT-LAMP time. FIG. 3.2D shows the calibration curve between the threshold time (Ct) and influenza A virus TCID₅₀ in each reaction (in log scale).

FIG. 3.3A shows pictures of the detection units taken under room light after RT-LAMP assay at 62.5° C. for 25 min; FIG. 3.3B shows the device of 3.3A under blue LED and FIG. 3.3C shows gel electrophoresis of those samples in 3.3A. These results are from SARS-CoV-2 detection. FIG. 3.3D shows pictures of the detection units taken under room light after RT-LAMP assay at 62.5° C. for 25 min. FIG. 3.3E shows the device of 3.3D under blue LED and FIG. 3.3F shows gel electrophoresis of those samples in 3.3D. These results are from influenza A virus detection.

FIG. 3.4A shows pictures of the detection units under room light using SARS-CoV-2 assay while samples contain SARS-CoV-2 RNA (S), influenza A virus RNA (F), CoV-OC43 RNA (O), and no RNA (N), respectively. FIG. 3.4B is the same devices as 3.4A under blue LED. FIG. 3.4C shows pictures of the detection units under room light using influenza A virus assay while samples contain influenza A virus RNA (F), SARS-CoV-2 RNA (S), CoV-OC43 RNA (O), and no RNA (N), respectively. FIG. 3.4D provides pictures of the same device in FIG. 3.4C under blue LED.

FIG. 3.5A shows simultaneous detection of heat-inactivated SARS-CoV-2 and influenza A H1N1 viruses using the 2-plex VLEAD device in accordance with embodiments of the present disclosure. FIG. 3.5B shows multiplexed detection of SARS-CoV-2 and influenza A H1N1 viruses in environmental sample #2.

FIG. 4.1A provides an exploded view of the VLEAD, in this instance a singleplex modular unit that can be attached to additional singleplex units to form a multiplex VLEAD. FIG. 4.1B shows an assembled real-time detection platform consisting of a pencil-shaped microscope and 3D-printed chambers for a heater, battery, and integrated electrical circuit in accordance with embodiments of the present disclosure. FIG. 4.1B is an alternative to the combination of FIG. 1.4 and FIG. 1.5; the former is capable of real-time detection while the latter is for end-point detection.

FIGS. 4.2A-4.2C are camera images of the real-time detection platform components in the portable analysis device shown in FIG. 4.1B. FIG. 4.2A shows a 3D-printed cradle (base); FIG. 4.2B show 3D-printed caps for three chambers for the heater, battery, and integrated electrical circuit; and 4.2C shows electrical components of the detection platform without the chamber caps.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, microbiology, material science, and the like, which are within the skill of the art.

The following examples are put forth so as to provide those of ordinary skill in the art with a complete disclosure and description of how to perform the methods and use the devices and methods disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### Definitions

The term “microorganism” or “microbe,” as used herein, refers to a small (often, but not always, microscopic) organism that is typically, but not exclusively, single cellular, and includes organisms from the Kingdoms bacteria, archaea, protozoa, and fungi. Viruses are also classified as microorganisms in the present disclosure document even though they are not living organisms by themselves.

### Abbreviations

VLEAD, a valve-enabled lysis, paper-based RNA enrichment, RNA amplification device; POC, Point of Care; RT-PCR, reverse transcription polymerase chain reaction; RT-LAMP, reverse transcription loop-mediated isothermal amplification; NAAT, nucleic acid amplification tests.

### General Discussion

The “gold-standard” test recommended by the World Health Organization (WHO) and the U.S. Centers for Disease Control and Prevention (CDC) for the detection of SARS-CoV-2 is reverse transcription polymerase chain reaction (RT-PCR). This test is generally performed in a laboratory setting, takes several hours to complete, and requires expensive equipment and highly trained personnel. It often takes 1-2 days from sample collection to result-reporting because the collected sample needs to be transported to a laboratory, where they are often first stored frozen before being processed using nucleic acid extraction and purification protocols prior to RT-PCR. The sample preparation protocols also require laboratory equipment such as centrifuges, and often include spin columns for a solid phase extraction step. Transportation and storage of samples are not needed if POC devices are used, reducing the sample-to-answer time. POC devices must include the sample preparation steps and require no laboratory equipment, thus facilitating their use by non-specialized personnel. It should be noted that sample preparation steps have been eliminated from some SARS-CoV-2 detection systems⁴⁻⁵. However, these approaches can reduce detection sensitivity. For example, Dao Thi et al. showed that the detection sensitivity reduced from 97.5% with sample preparation to 86% without sample preparation in their reverse transcription loop-mediated isothermal amplification (RT-LAMP) assay systems⁵.

Some POC devices are based on antigen tests, such as Panbio™ Covid-19 Ag Rapid Test device (Abbott)⁸, and they are often used for rapid screening. However, the limited sensitivity of antigen tests can result in false negatives, leading to detrimental consequences during pandemics⁷. Efforts have been made to increase their detection sensitivity including various biosensors^{8,9}. It is generally recognized that nucleic acid amplification tests (NAAT) are more sensitive and they are often preferred over antigen tests, especially for reducing disease transmission by asymptomatic and pre-symptomatic persons¹⁰. Moreover, NAAT are easily adapted for the detection of different viruses^{11,12} and can thus be configured to allow users to test for SARS-CoV-2, influenza, and other respiratory viruses at the same time. Multiplexed nucleic acid diagnostic tools will be useful to help control the COVID-19 pandemic and for influenza outbreaks, as well as for patient care¹³.

The first two SARS-CoV-2 NAAT POC kits approved for emergency use authorization (EUA) by the U.S. Food and Drug Administration (FDA) are Lucira All-In-One Test Kit and Cue Test for Home and Over the Counter (OTC) Use¹⁴. The Lucira kit combines RT-LAMP with colorimetric detection, while the Cue test employs an unspecified isothermal amplification followed by electrochemical detection. Many POC testing platforms have been reported for influenza virus detection, including RT-LAMP devices¹⁵⁻¹⁷, microfluidics-based RT-PCR¹⁸, and other nucleic acid isothermal amplification assays¹⁹. Similarly, POC platforms for SARS-CoV-2 detection have been developed to incorporate RT-PCR²⁰, RT-LAMP-^{5,21,22}, other amplification methods.^{23,24}, and those combined with CRISPRs²⁵⁻²⁷.

However, little effort has been exerted for the development of POC platforms for simultaneous detection of SARS-CoV-2 and other respiratory viruses such as influenza viruses. Chung et. al used the BD MAX™ system¹³, and Mostafa et. al employed the Cepheid Xpert®²⁸ for detection of SARS-CoV-2 and influenza viruses in health care settings. Others have developed portable platforms for detection of these viruses, but all of these require laboratory equipment for sample preparation^{29,30}. Ji et. al reported a centrifugal RT-PCR microfluidic device for SARS-CoV-2 and influenza virus detection, but the device needs an instrument for spinning, thermocycling, and optical detection steps³⁰. To our knowledge, there is no POC device available for simultaneous detection of SARS-CoV-2 and influenza viruses that includes all the necessary steps from sample-to-answer in a portable testing platform without the need of bulky or expensive laboratory equipment that require power outlets.

In accordance with the purpose(s) of the present disclosure, as embodied and broadly described herein, embodiments of the present disclosure, in some aspects, relate to devices for preparation of a sample for RNA detection, devices for detection of RNA in a sample, and methods for preparation and/or detection of RNA in a sample.

The present disclosure includes devices for preparing samples for the detection of RNA or other nucleic acids. Described herein are several embodiments of a valve-enabled lysis, paper-based RNA enrichment, RNA amplification device (VLEAD) for duplexed and multiplexed detection of multiple viruses (e.g., SARS-CoV-2 and influenza virus) or other microorganisms with no requirement for pipets and power outlets during device operation.

While the examples in the present disclosure use RT-LAMP, the devices can be used with LAMP to target DNA viruses as well. Where RNA and RT-LAMP are discussed, DNA and LAMP can be substituted and appropriate buffers and/or reagents used. The term amplification unit can refer to an RT-LAMP or LAMP unit.

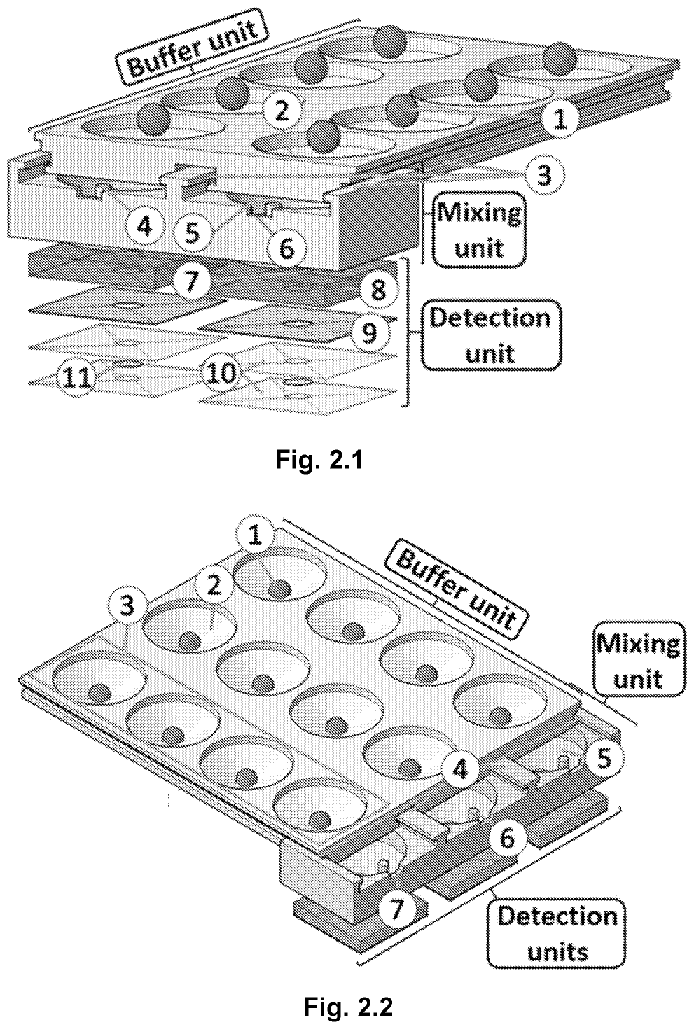

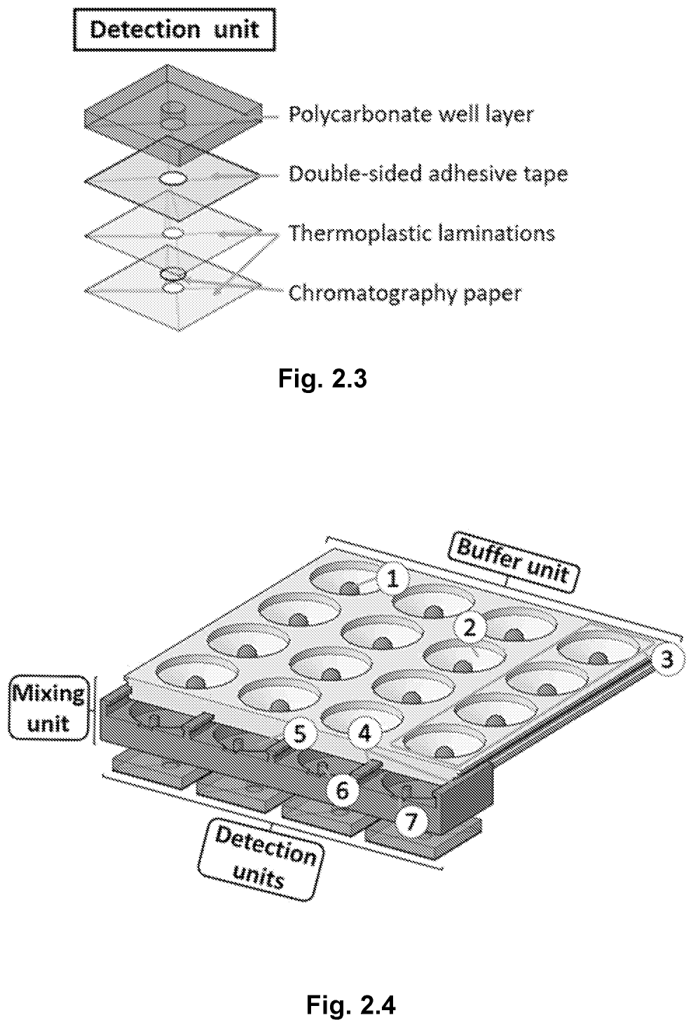

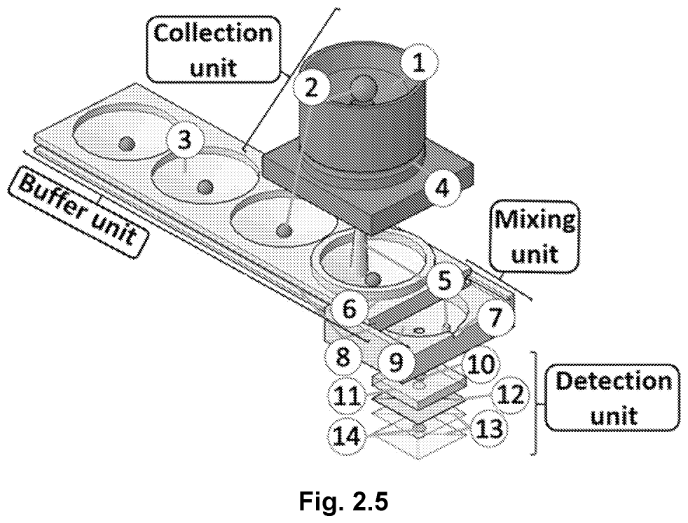

Advantageously, the device can be used to prepare samples for, and subsequently detect, numerous viruses and/or microorganisms simultaneously. The device can include at least two parallel buffer units. Each buffer unit can include a plurality of buffer wells arranged in a row. Each buffer well has a ball valve in the bottom of the well. An RT-LAMP unit can be coupled to an end of each buffer unit to form a singleplex unit, where the RT-LAMP unit also includes a buffer well and ball valve. The RT-LAMP unit may be stored in a low temperature environment (e.g., with cold packs) before it is attached to VLEAD immediately before use. The singleplex units can be modular such that the device can be expanded to accommodate from about 2 to about 10 singleplex units. In an alternative embodiment, the device can be manufactured such that the singleplex units are permanently connected (e.g., integral to the device) as shown in FIGS. 2.1, 2.2, and 2.4.

By combining modular singleplex units or providing a single multiplex unit, multiple tests can be run simultaneously on samples obtained from a single patient or multiple patients. For example, a patient sample could be tested for influenza and SARS-CoV-2 at one time. Similarly, a patient sample could be tested for multiple diseases associated with viruses such as Mayaro virus (MAYV), Chikungunya virus (CHKV), Dengue virus (DENV), and Zika virus (ZIKV) simultaneously at the point of care. Similarly, a patient sample could be tested for multiple subtypes of one virus, such as DENV-1, DENV-2, DENV-3, and DENV-4 simultaneously at the point of care. Similarly, a patient sample could be tested for multiple variants of one virus, such as SARS-CoV-2 alpha, beta, delta, and omicron simultaneously at the point of care. A single technician can process multiple samples, increasing the efficiency of testing. Traditionally, each sample must be handled individually, which either requires multiple technicians or a single technician taking time to process each sample separately. The multiplex system described herein can reduce the number of trained technicians needed to process samples and speed the processing time.

Each singleplex unit can be filled with buffers to target a specific virus or microorganism. For example, in the duplex device in FIG. 2.1, a first row can be configured to target SARS-CoV-2, a second row configured to target influenza virus. For example, in the fourplex device in FIG. 2.4, a first row can be configured to target SARS-CoV-2, a second row configured to target influenza virus, a third row configured to target Zika virus, and a fourth row configured to target Dengue virus. A sample from a single patient can be divided between the two rows to test a single patient for both viruses at once in the case of the duplex device. In another example, each row in the device can be configured to target the same virus or microorganism and a sample from a different patient or source can be introduced to each row. In this manner, samples from multiple patients could be processed at one time.

While the examples of the present disclosure mainly relate to the detection of viruses such as SARS-CoV-2 and influenza virus from a patient, the device can be used for other viruses and microorganisms including but not limited to Zika virus, Dengue virus, Chikungunya virus, Ebola virus, hepatitis A virus, hepatitis B virus, hepatitis C virus, human immunodeficiency viruses (HIV), Mayaro virus, Escherichia coli (E. coli), enterococci, Salmonellae, Streptococcus pneumoniae, Staphylococcus aureus and Pseudomonas aeruginosa. The device can also be used to detect pathogenic and nonpathogenic microorganisms such as from air, water, or food samples. In some embodiments the samples can be any source in which a target RNA source may be present such as blood, urine, saliva, mucosal swabs, aerosolized respiratory particles and the like.

Advantageously, sample collection, processing, and detection can be performed all at the point of collection if desired. By using RT-LAMP or other isothermal amplification methods, samples can be detected in about an hour under ideal conditions. Both the device and the heating unit used to amplify the target RNA or DNA sample are small, portable, and can be manufactured to be disposable or sterilizable for reuse. The devices and methods herein are highly sensitive, having a level of detection for specific targets as low as about 1 genomic equivalent. For example, SARS-COV-2 can be detected in a sample including about 2 genomic copies and influenza virus can be detected in a sample including about 6 genomic copies per sample, where the sample is 140 microliters. Bacteria such as E. coli can be detected at levels as low as about 3 genomic copies of DNA per 140 microliters of liquid sample or per 1000 microliters of liquid sample with a modified device.

The device can be provided pre-loaded with appropriate reagents for a target virus or microorganism or the buffers can be added such as at a lab or point of collection. In some embodiments, leakage of preloaded buffers (e.g., during shipping or storage) is prevented by sealing the ball valve with a biocompatible substance (such as wax), wherein the substance is breakable when the ball valve engages with the mixing unit to release the liquid when needed.

This is just one way to prevent possible ball movement during shipping for example. Wax is one way to limit any movement temporarily. In another embodiment, the device can include a lid having a pin on the underside, wherein the pin presses against the ball during shipping or the like. The lid is removed before using the device, and the pin is removed as a part of the lid.

The device can further include a mixing unit associated with each singleplex unit, wherein the mixing unit comprises a mixing well and a pin, wherein the mixing unit is slidably connected to a bottom of the buffer unit such that the mixing unit can be moved along a length of the buffer unit to align with each buffer well in turn, wherein when the mixing unit is aligned with a buffer well the pin engages with the ball valve to release buffer from the buffer well into the mixing well. The device can further include a detection unit removably coupled to the bottom of each mixing unit to receive fluids from the mixing well a drain unit removably coupled to the bottom of each detection unit to receive fluids from the detection unit.

The detection unit can include an absorbent layer, wherein the absorbent layer is selected from chromatography paper, cellulose paper (e.g. an FTA™ card) and glass microfiber paper. Further details of the detection unit are provided in the examples.

The first well of each buffer unit can receive a sample. As described above, the sample can be the same for each buffer unit or not the same for each buffer unit.

The drain unit can include a tube compatible with a syringe, such that buffer flowed from the wells through the buffer unit, mixing unit, and detection unit can be extracted from the drain unit by the syringe. In other embodiments, the tube can be connected to a vacuum or other means to facilitate draining or suction.

In some embodiments, each buffer unit comprises four buffer wells and each RT-LAMP unit comprises a single well.

In some embodiments, the detection unit can include a heating unit, that contains a seat for one detection unit or multiple detection units. The heating unit provides a temperature optimized for amplification of nucleic acids (e.g., about 57° C. to 67° C.). In some embodiments, the heating unit includes at least one cartridge heater. Advantageously, the heating unit can be powered by batteries or a portable electronic device such as a mobile phone or laptop, such that no power source is needed.

In some embodiments, the detection unit can include a portable analysis device, where the portable analysis device includes the heating unit. The analysis device can serve as a base for the detection unit. The portable analysis device can be powered by a battery and include a temperature control circuit. The portable analysis device can include a stand for an imaging apparatus (e.g., a microscope) or both an imaging apparatus and the stand. In some embodiments, the microscope is a digital pencil type, and, the heating unit has a cap that provides a seat for the detection unit. In some embodiments, the microscope can be replaced by a CCD (charge-coupled device) camera, a CMOS (complementary metal oxide semiconductor) sensor, or other image-acquiring devices, especially when multiple detection units need to be imaged simultaneously. The portable analysis device can be used to acquire data, such as fluorescence imaging data, and can be connected to an external device such as a computer or smartphone for further data analysis.

In some embodiments, the imaging apparatus detects a signal of nucleic acid amplification in the detection unit in real time, leading to quantitative information about viral load (rather than binary results at the end point). Advantageously, the portable analysis device (or real time detection platform) is light and compact, enabling nucleic acid amplification and detection at the point of care or in the field.

In some embodiments, the sample can be provided to the singleplex unit via a sample collection unit or system containing a sample (such as a sample collected from a patient or an aerosol sample collected from a room, building or an open space into a liquid or a medium). The sample collection can have a sample well having a ball valve in a bottom of the sample well, similar to the ball valves in the wells of the buffer unit. The sample collection unit can be placed on top of the first buffer well so that the first buffer well receives the bottom of the sample collection unit. The ball valve of the collection unit is engaged to release contents of the sample collection unit into the buffer well.

Embodiments of the present disclosure include a multiplex device for detecting RNA from an RNA source as above, wherein the device includes at least two singleplex units. An RNA source and a first buffer is provided to a first buffer well, a second buffer is provided to a second buffer well, a third buffer is provided to a third buffer well, a fourth buffer is provided to a fourth buffer well and an RT-LAMP buffer is provided to the RT-LAMP buffer well.

A mixing unit associated with each singleplex unit, wherein the mixing unit comprises a mixing well and a pin, wherein the mixing unit is slidably connected to a bottom of the buffer unit such that the mixing unit can be moved along a length of the singleplex unit to align with each buffer well in turn beginning with the first buffer well and ending with the RT-LAMP unit. When the mixing unit is aligned with a buffer well, the pin engages with the ball valve to release buffer from the buffer well into the mixing well so that cDNA is produced in the mixing unit from the RNA source after the mixing unit engages with the RT-LAMP unit A detection unit is removably coupled to the bottom of each mixing unit to receive buffer and nucleic acid from the mixing well, wherein the nucleic acid is collected and amplified in the detection unit. A drain unit can be removably coupled to the bottom of each detection unit to extract buffer from the detection unit.

In some embodiments, the detection unit can be removed from the buffer unit and analyzed to determine whether the liquid received by the detection unit contains nucleic acid from a target virus or microorganism.

Each singleplex unit and associated detection unit can be configured to detect the same target virus or microorganism or a different target virus or microorganism.

In some embodiments, the device includes two singleplex units such that the device can detect both SARS-CoV-2 virus and influenza virus.

Embodiments of the present disclosure also include methods for preparing a sample to be used for detecting RNA from an RNA source. The RNA source can be a sample as described above. The method can include placing a first liquid sample in at least a first well of a first buffer unit of an RNA detecting apparatus as described above, placing at least a second, third and fourth liquid in at least second, third and fourth wells, respectively, of the first buffer unit of the RNA detecting apparatus. The method further includes placing a second liquid sample in at least a first well of a second buffer unit of the RNA detecting apparatus, and placing at least a second, third and fourth liquid in at least second, third and fourth wells, respectively, of the second buffer unit of the RNA detecting apparatus. An RT-LAMP unit coupled to an end of each buffer unit after the fourth well, the RT-LAMP unit comprising a well and a valve mechanism. In some embodiments, the method can be used to detect DNA from a DNA source when the RT-LAMP unit is a LAMP unit. Where “nucleic acid” is used herein, nucleic acid can refer to RNA or DNA depending upon the source material and the stage in the process.

As above, a mixing unit is slidably coupled to a bottom of each buffer unit allowing a user to create relative movement between the mixing unit and the buffer unit by exerting a manual force on at least one of the buffer unit and the mixing unit; and exerting the manual force on at least one of the buffer unit and the mixing unit to slide the mixing unit along the buffer unit to cause the first, second, third, and fourth wells of the buffer unit to come into temporary alignment with the mixing unit for respective time periods, followed by temporary alignment with the RT-LAMP well. A valve mechanism in each of the wells causes the first, second, third and fourth liquids to be released from the first, second, third and fourth wells, respectively, into the mixing unit, in turn, when the manual force is exerted to cause the second, third and fourth wells to come into temporary alignment with the mixing unit for respective time periods such that each liquid sample is prepared for nucleic acid amplification as it combines with the buffers from the wells in the mixing unit. A detection unit is coupled to a bottom of the mixing unit and receives fluid from the mixing unit.

In some embodiments, the first well of the first buffer unit contains a lysis buffer, the second well of the first buffer unit contains a binding buffer, the third and fourth wells of the first buffer unit each contain a washing buffer, and the RT-LAMP unit contains an RT-LAMP buffer specific to a first target RNA sample. Similarly, the first well of the second buffer unit contains a lysis buffer, the second well of the second buffer unit contains a binding buffer, the third and fourth wells of the second buffer unit each contain a washing buffer, and the RT-LAMP unit contains an RT-LAMP buffer specific to a second target RNA sample. As the mixing unit for a respective buffer unit is moved from well to well, cells in each liquid sample are lysed by the lysing buffer to release RNA, the released RNA in the sample is bound by the binding buffer, and the RNA is purified by the washing buffers and collected on a detection pad in the detection unit.

In some embodiments, the first liquid sample and the second liquid sample are from the same source, and the first target RNA sample (e.g. the virus or microorganism desired for detection) and the second target RNA sample are different.

In some embodiments, the method includes amplifying the nucleic acid by sliding the mixing unit from alignment with the fourth well to the RT-LAMP unit to release RT-LAMP buffer or LAMP buffer from the RT-LAMP unit into the mixing unit, such that the RNA or DNA is amplified on the detection pad in the detection unit.

The collected buffer can be drained from the detection unit via a drain unit coupled to the bottom of the detection unit.

In some embodiments, each detection unit can be removed from the apparatus and analyzed to determine whether the liquid received by the detection unit contains target RNA from a target virus or microorganism.

In some embodiments, the analysis is performed by heating the detection unit on a heating unit to amplify the RNA extracted from the sample.

In some embodiments, amplified product can be detected calorimetrically by adding a dye such as SYBR green or SYTO-9. The color change can be observed by naked eye or recorded by a device such as a smart phone camera.

In some embodiments, the dye can be added before isothermal amplification (e.g., as a part of RT-LAMP mix).

In some embodiments, color change can be enhanced by using a UV flashlight, which can generate fluorescence.

In some embodiments, fluorescence signal can be filtered to increase the detection sensitivity.

In some embodiments, fluorescence signal can be detected simultaneously while the amplification takes place, resulting in real time detection.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

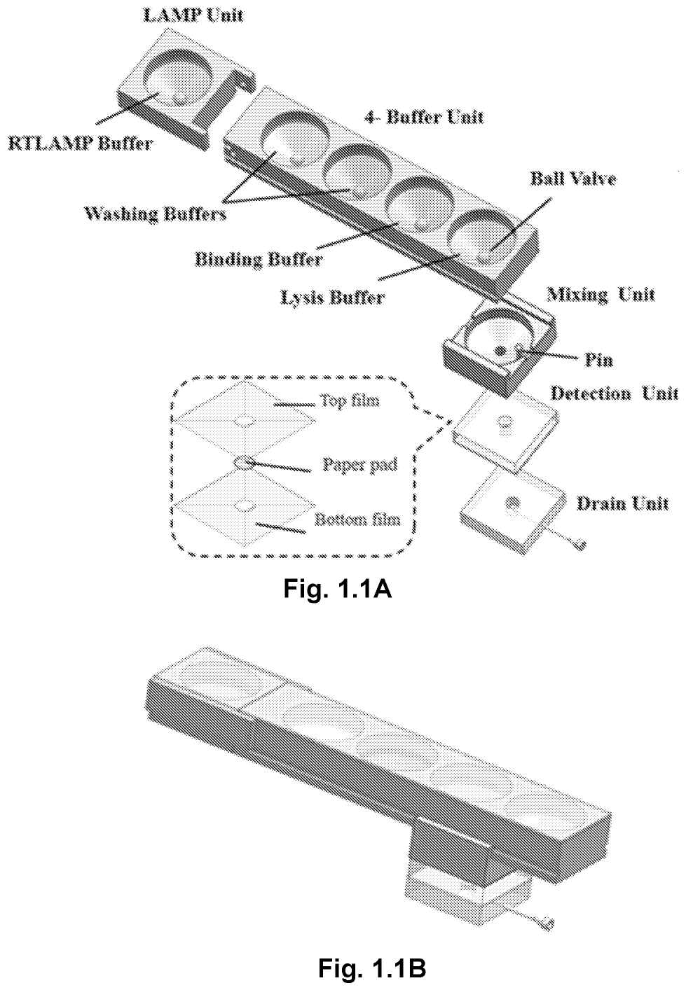

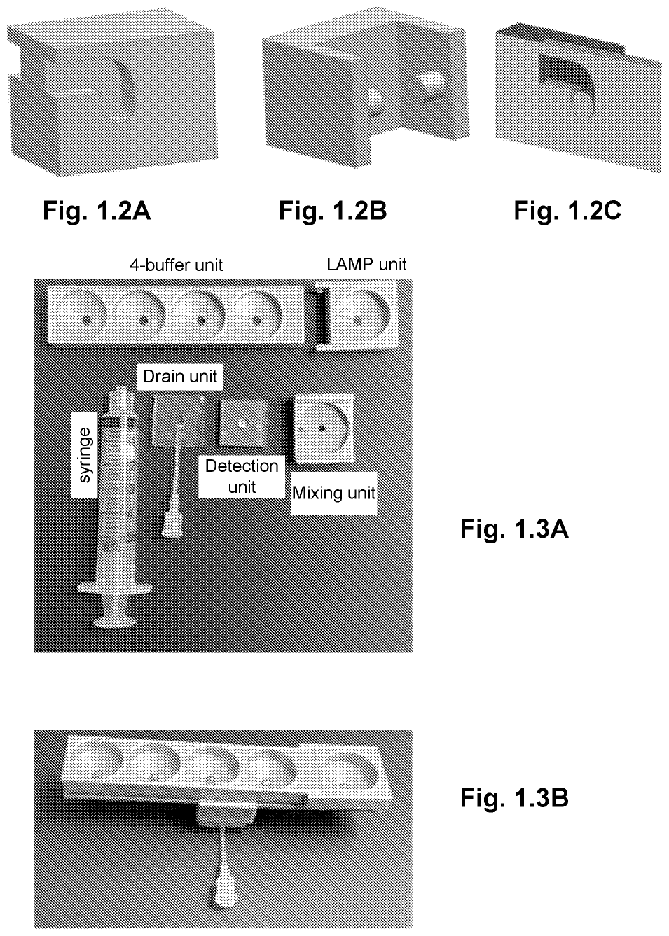

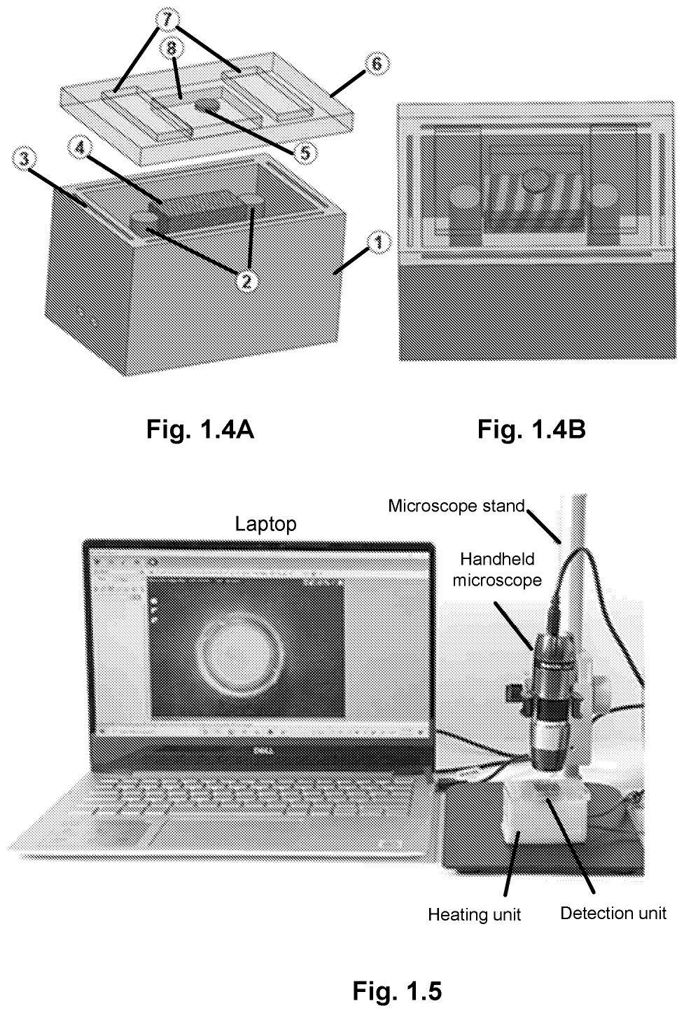

FIGS. 1.1A-1.1B provide a perspective view showing components of an embodiment of a multi-plex VLEAD apparatus. As shown in FIG. 1A, the VLEAD apparatus comprises a 4-buffer unit, a LAMP unit, a mixing unit, a detection unit, and a buffer drain unit (referred to hereinafter as “drain unit”). The 4-buffer unit was designed for storing lysis buffer, binding buffer, and two washing buffers. The LAMP unit is used for storing the RT-LAMP buffer (or a LAMP buffer when a DNA source is targeted). While performing the test, the RT-LAMP unit will be integrated with the 4-buffer unit using a sliding mechanism (see FIGS. 1.2A-1.2C). The mixing unit consists of a pin near a side wall, a mixing well at the center, and a protrusion at the very end of the mixing unit. The pin of the mixing unit is used to lift up the ball-valves used to prevent the reagent's discharge from the buffer units until desired. The ball-valve mechanism was previously disclosed (US 2021 0230533 A1). FIG. 1.1B shows an assembled view of the VLEAD.

In an embodiment, the combined 4-buffer unit and the LAMP unit (referred to hereinafter as “buffer unit”) is slidably coupled with the mixing unit via a sliding mechanism (FIGS. 1.2A-1.2C). The sliding mechanism consists of a pair of rails on the mixing unit that engages a pair of grooves on the buffer units.

A commercial 3D printer, the Ultimaker 3 (Ultimaker, Geldermalsen, Netherlands) was used to fabricate the buffer unit and the mixing unit. The devices were printed from polylactic acid (PLA) as print material and with polyvinyl alcohol (PVA) as support material. The print layer height was set to 0.06 mm and the infill density was set to 100%. The exploded view and the complete assembly of the VLEAD device is shown in FIGS. 1.3A and 1.3B, respectively. As can be envisioned by one of ordinary skill in the art, any manufacturing means and materials suitable for use in sample collection can be used.

The detection unit is a paper-based amplification device including a polycarbonate container, a double-sided adhesive tape, and a laminated paper pad. The polycarbonate container was made of a 3 cm thick, 2 cm-by-2 cm transparent square polycarbonate sheet with a circle container machined at its center. The laminated paper-pad is a Whatman™ chromatography paper (Fisher Scientific, Pittsburgh, PA, USA) that was cut in 3.5 cm using a steel puncher. Two layers of thermoplastic films were made by cutting 75-μm thick polyester thermal bonding lamination films (Lamination Plus, Keysville, UT, USA) using a Graphtec Craft Robo-S cutting plotter (Graphtec Corporation, Yokohama, Japan). The paper-pads were then sandwiched between the films and then aligned and passed through a heated laminator (GBC Catena Roll Laminator, GBC, Lake Zurich, IL, USA), which was set at a rolling speed of “1” and the temperature and 220° F., respectively. The laminated paper pad was then attached to the polycarbonate container via a piece of 3M 9087 white bonding tape (R. S. Hughes, Sunnyvale, CA) to form the RNA detection unit.

The drain unit, which was made of polydimethylsiloxane (PDMS), was designed to improve the draining of the buffers. The buffer drain unit is a 2 mm-thick, 4 mm-diameter circular chamber fabricated at the center of 2 cm-by-2 cm cured PDMS. A 500-μm inner diameter tube also designed to connect the interior of the chamber to a syringe, which is used to suck the buffers out of the chamber by pulling its plunger out (See FIGS. 1.3A and 1.3B). To fabricate the drain unit, a mold was fabricated in a transparent square polycarbonate sheet. Then the tube was placed in the chamber and the model was casted by PDMS. After the PDMS was cured, the PDMS was peeled off the mold. The drain unit was then attached to the bottom side of the detection unit using a double-sided adhesive tape.

The mixing unit is integrated with the detection unit by inserting the bottom protrusion of the mixing unit into center hole of the detection unit that is designed for the collection of the RT-LAMP buffer and RNA amplification, as shown in FIG. 1.1B.

### Heating Unit:

FIG. 1.4A is an exploded perspective view of a custom-made heating unit for RT-LAMP incubation; FIG. 1.4B shows the assembled unit. As shown in FIG. 1.4A, the heating unit comprises a container (1) that is filled with paraffin wax, two cartridge heaters (2), two copper foam pieces (4 and 5), and a cap (6). The cap is designed to seal the container as well as provide a seat for the detection unit. The cartridge heaters need ˜5 V power source and can be powered by a cellphone or laptop or batteries. The copper foam block is used to transfer the heat, generated by the cartridge heaters, to the center of the heating unit, where the detection unit is located in a detection unit seat (8). Paraffin with 65±2° C. melting temperature is used for providing an isothermal temperature of between 63-67° C., the required temperature for the RT-LAMP assay reaction. The volume of paraffin expands into the expansion room (7) when heated. The container's width, length, and height are 3.5 cm, 5 cm, and 3 cm, respectively. The side walls comprise two 2 mm thick layers with a 1 mm air gap (3) in between for improving the thermal resistance of the walls.

A commercial 3D printer, the Ultimaker 3 (Ultimaker, Geldermalsen, Netherlands) was used to fabricate the container. The devices were printed from polycarbonate (PC) as print material. The print layer height was set to 0.06 mm and the infill density was set to 20%. Two 6 mm-diameter air balloons were used to accommodate the expanded liquid paraffin.

Polycarbonate sheet was used to fabricate the heating unit cap. A 2.02 mm-by-2.02 mm square chamber was machined at the center of the polycarbonate cap as the detection unit seat. For transferring the heat to the detention unit well, a 6 mm-diameter, 1 mm-depth cylindrical well was machined at the center of the chamber and filled with a piece of a copper foam. This reduces the thermal resistance between the heating unit and the reaction well of the detection unit. This custom-made heating unit allows for real-time observation of the RNA amplification using either a simple UV light or a handheld microscope (optical-detection and image-analysis module).

Alternative heating mechanisms such as thermal resistors or printed microheaters may be used to achieve the targeted temperature range of 57° C. to 67° C.

### Real-Time Optical-Detection Module:

A Real-time RT-LAMP-enabled diagnostic platform, or portable analysis unit, was developed using a portable digital microscope (Model M4117MT-G2FBW, Dunwell Tech Inc., Torrance, CA, USA) equipped with green fluorescence using 465 nm excitation light and a 510-545 nm emission bandpass filter range. The microscope can be actuated by a computing device such as a laptop or smartphone. The computing device can be used for monitoring the real time RNA amplification by bare human eye.

In the embodiment shown in FIG. 1.5, the portable analysis unit consists of a computing device, a handheld microscope with its stand, and the heating unit. The heaters of the heating unit are powered by a device such as a laptop or a cellphone. In some embodiments, the same device may also control the microscope. After sample preparation in the detection unit, the detection unit can be separated from the mixing unit and placed on the detection unit seat designed in the heating unit cap for 25 minutes. A transparent tape is used to press the detection unit to its place, eliminating the gap between them.

In tests of the system, the heating unit was pre-heated for 35 minutes, such that the paraffin wax was heated to 62° C. DinoCapture 2.0 software (DinoLite®) was used to record time-lapsed video. ImageJ software is then used to analyze the fluorescence intensity emitted from the reaction well. Alternatively, a software can directly plot the video signal as a function of time.

### Example 2

Another embodiment of the VLEAD device is a duplex device for the simultaneous detection of two types of viruses (e.g., SARS-CoV-2 and Influenza A viruses). The device includes two sets of buffer units in the top, a mixing unit in the middle, and a detection unit at the bottom (FIG. 2.1). The detection units are connected at the bottom of each mixing well by a protrusion, and the mixing unit is connected to the buffer unit through a sliding mechanism, which also allows for manipulation of valves. In addition, the 2-plex device has another sliding mechanism in the middle for extra support and better printing quality in comparison with the 1-plex device that has sliding tracks on the two sides only (US patent publication US 2021 0230533, the disclosure of which is incorporated herein by reference). Fluid-control valves based on stainless steel balls placed in each buffer well are used for sequential release of the reagents from the buffer unit to the mixing unit for sample preparation as reported in patent publication US 2021 0230533. The indicated parts are: Ball for valving (1), buffer well (2), sliding mechanism (3), pin for valving (4), mixing well (5), gap for the balls that are not pushed up by the well edge during sliding (6), protrusion for connecting detection unit to mixing well (7), well layer (8), adhesive tape (9), thermoplastic films (10), and paper (11).

FIG. 2.2 shows the design of the 3-plex VLEAD for the simultaneous detection of three different viruses. The device consists of three parallel sets of buffer unit in the top, mixing unit in the middle, and detection unit at the bottom. For each set, the buffer unit contains 4 buffer wells (2) designated to a lysis buffer, a binding buffer, and two wash buffers, respectively. First, the sample is mixed with the lysis buffer for virus lysate, then immediately mixed with the binding buffer as well, to bind the released RNA onto the paper substrate in the detection unit. Finally, the two wash buffers are released one at a time to purify the collected RNA. These buffers are sequentially release from the buffer unit to the mixing unit, which are connected through sliding slots mating with sliding mechanism (4) on the sides and between each set, using ball-based valves (1). One stainless steel ball is placed at the bottom of each buffer well preventing the reagents from going down until desired. These balls protrude slightly from the bottom of the buffer unit, and are actuated by sliding the mixing unit under it, where each mixing well (5) contains a pin (6) that reaches the bottom surface of the buffer unit so that when the pins align with the balls, they lift them up, discharging the respective reagents (3) into the mixing wells. The gap (7) is for the balls that are protruded slightly; without this gap, the ball will be pushed up by the well edge during sliding. Then, each detection unit is connected at the bottom of each mixing well by a protrusion. The detection unit contains a chromatography paper laminated between two thermoplastic films and attached at the bottom of a polycarbonate well layer using a double-sided adhesive tape as shown in FIG. 2.3. After this sample preparation process, these detection units are detached and prepared for nucleic acid amplification. If the device includes a drain unit, the drain unit can be removed from the detection unit.

FIG. 2.4 shows the design of a 4-plex VLEAD for the simultaneous detection of four different viruses. The device consists of four parallel sets of buffer unit in the top, mixing unit in the middle, and detection unit at the bottom, similar to the single-plex VLEAD device previously disclosed (US 2021 0230533 A1). For each set, the buffer unit contains 4 buffer wells (2) designated to a lysis buffer, a binding buffer, and two wash buffers. First, the sample is mixed with the lysis buffer for virus lysate, then immediately mixed with the binding buffer as well, to bind the released RNA onto the paper substrate in the detection unit. Finally, the two wash buffers are released one at a time to purify the collected RNA. These buffers are sequentially released from the buffer unit to the mixing unit, which are connected through sliding slots (4) on the sides and between each set, using ball-based valves (1). One stainless steel ball is placed at the bottom of each buffer well preventing the reagents from going down until desired. These balls protrude slightly from the bottom of the buffer unit, and are actuated by sliding the mixing unit under it, where each mixing well (5) contains a pin (6) that reaches the bottom surface of the buffer unit so that when the pins align with the balls, they lift them up, discharging the respective reagents (3) into the mixing wells. The gap (7) is for the balls that are protruded slightly; without this gap, the ball will be pushed up by the well edge during sliding. Then, each detection unit is connected at the bottom of each mixing well by a protrusion. The detection unit contains a chromatography paper laminated between two thermoplastic films and attached at the bottom of a polycarbonate well layer using a double-sided adhesive tape. After this sample preparation process, these detection units are detached and prepared for nucleic acid amplification.

FIG. 2.5 shows the design of a modified VLEAD for the interface connection with an aerosol collection system (related to U.S. Pat. No. 10,859,473, the contents of which are incorporated herein by reference). The device consists of a buffer unit in the top, a collection unit on top of the first well of the buffer unit, a mixing unit in the middle, and detection unit at the bottom. The device shown has an additional unit for aerosol sample collection, the collection unit, and additional features in the first well of the buffer unit for integration with the collection unit compared to the single-plex VLEAD device previously disclosed (US 2021 0230533 A1). For appropriate connection between the buffer unit and collection unit, the buffer unit is enlarged compared to the regular VLEAD, and thus, the mixing unit too for integration with the buffer unit. The collection unit contains a collection well (1), a ball for valving (2), and a ring connection (4). The collection unit contains a lysis buffer where the aerosol sample is directly dispensed, and in this case, the first well of the buffer unit does not contain any reagents as it is purely use for integration with the collection unit and discharge the lysed sample into the mixing unit. Then, the rest of the wells of the buffer unit (3) contain a binding buffer and two wash buffers for RNA enrichment and purification. The sample preparation is similar to the regular VLEAD except that the sample is directly collected in a lysis buffer but then, the same sequential release of reagents is performed. Sample/lysis/binding buffer mixture goes through the detection unit to bind RNA onto the paper pad (14) of the detection unit, and then, 2 wash buffers are used to purify the collected RNA. These buffers are sequentially released from the buffer unit to the mixing unit, which are connected through sliding slots (8) on the sides, using ball-based valves. The sample/lysis mixture is also released from the collection unit onto the buffer unit using a ball valve, however, this one is manipulated when the collection unit is detached from the aerosol collector and placed on top of the buffer unit in the insertion (6) around the first buffer well, which contains a large pin (5) to lift up the ball. The gap (7) is for the balls that are protruded slightly; without this gap, the ball will be pushed up by the well edge during sliding. One stainless steel ball is placed at the bottom of each buffer well to prevent the reagents to go down until desired. These balls protrude slightly from the bottom of the buffer unit, and are actuated by sliding the mixing unit under it, where the mixing unit contains a pin (5) that reaches the bottom surface of the buffer unit so that when the pin is aligned with a ball, it lifts it up, discharging the respective reagent into the mixing unit (9). Then, the detection unit is connected at the bottom of the mixing unit by a protrusion (10). This detection unit contains a chromatography paper (14) laminated between two thermoplastic films (13) and attached at the bottom of a polycarbonate well layer (11) using a double-sided adhesive tape (12). After this sample preparation process, the detection unit is detached and prepared for nucleic acid amplification.

### Example 3

The 2-plex or multiplex VLEAD integrates (1) paper-based sample preparation using ball-based valves for sequential delivery of reagents and (2) RT-LAMP in a commercially available, battery-operated coffee mug with (3) colorimetric detection. The device consists of two sets of components fabricated in one platform with each set dedicated to one type of virus. The sample preparation process starts by sliding a part of the device to open valves sequentially that discharge various reagents for RNA extraction and purification while collecting RNA onto a paper pad. The reagents needed for sample preparation are pre-loaded in the device, thus requiring no pipetting at POC. RNA collected on the paper pad is then amplified by RT-LAMP, followed by colorimetric detection. This 2-plex VLEAD is low cost and easy to use, providing results at point of care (POC) in much shorter time than RT-PCR, with similar sensitivity.

### Experimental Section

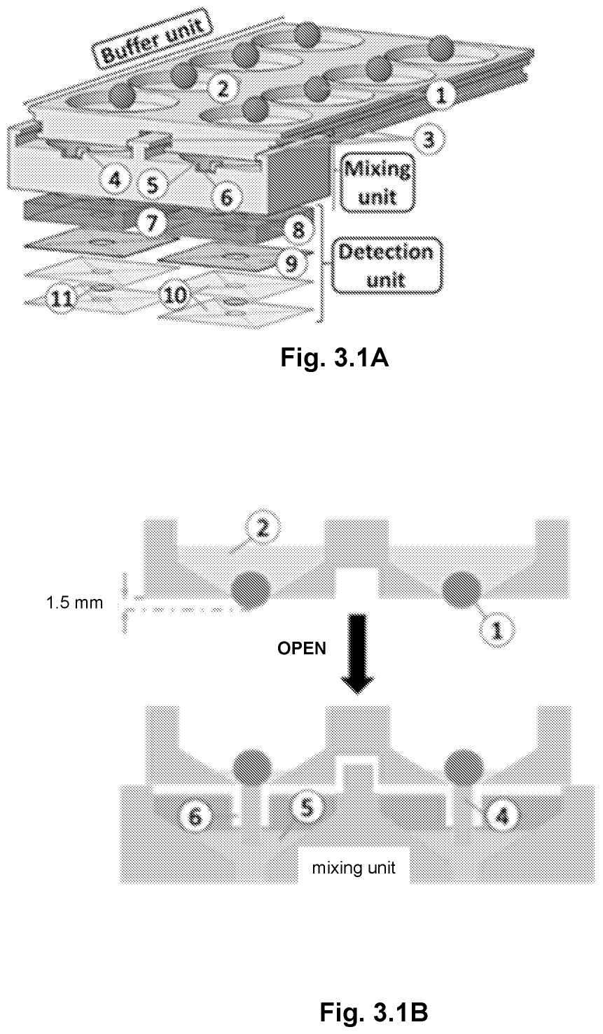

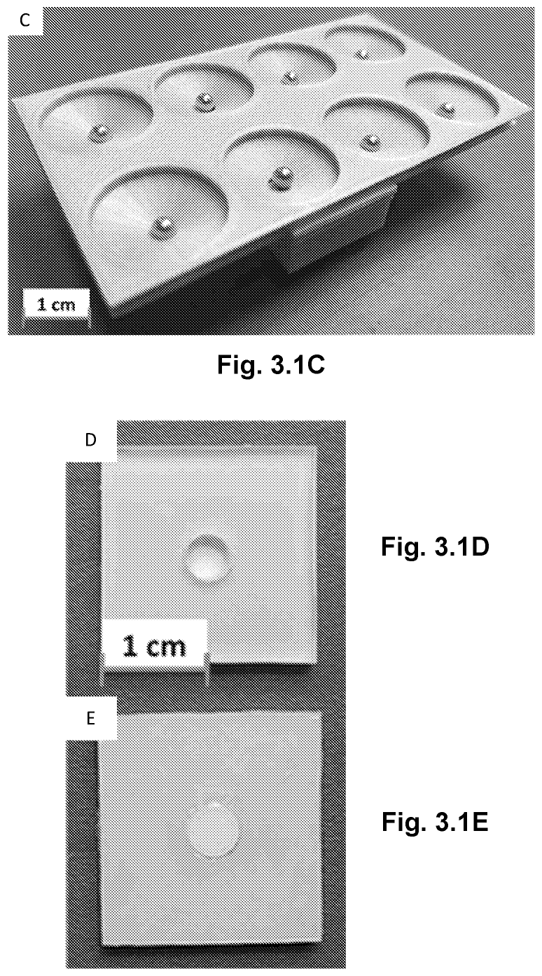

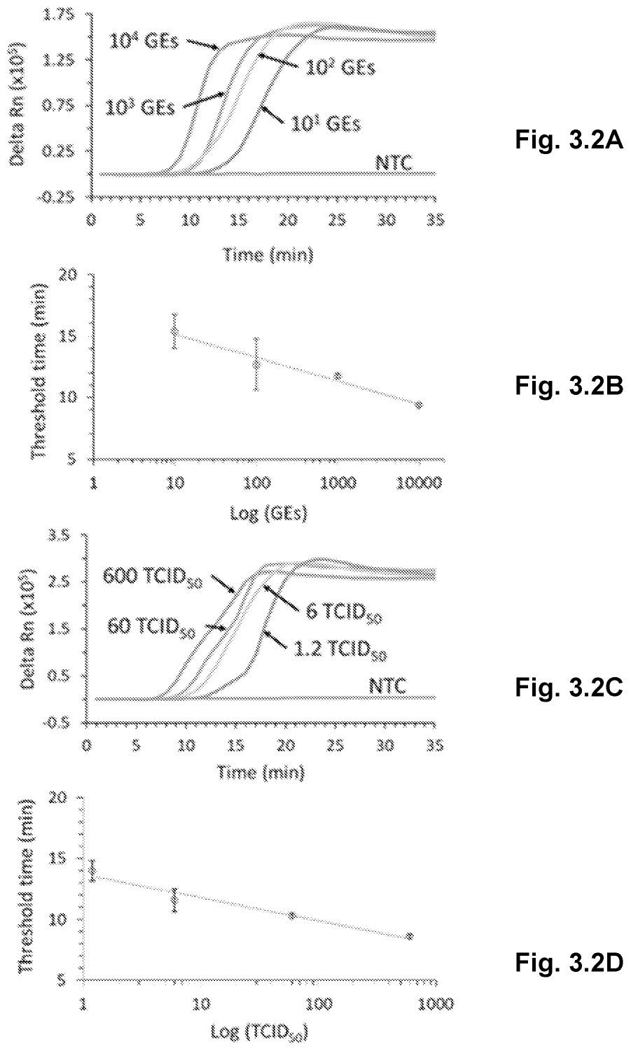

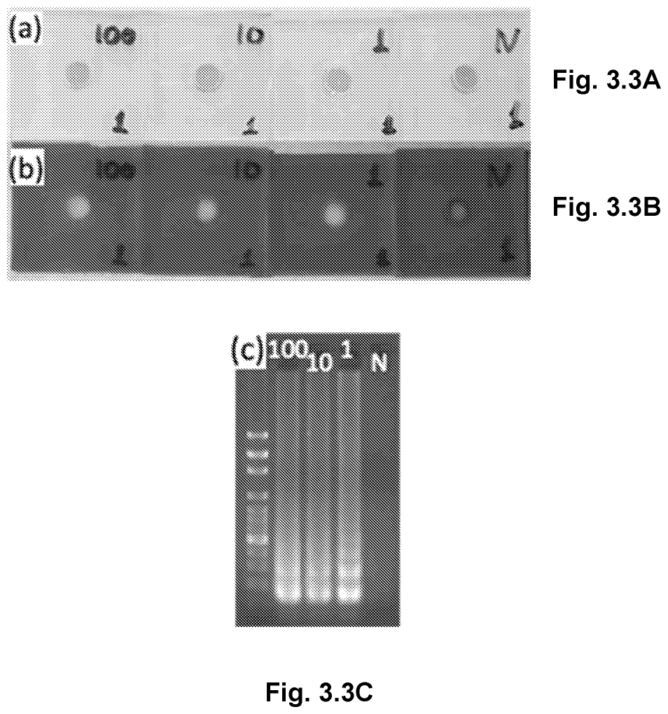

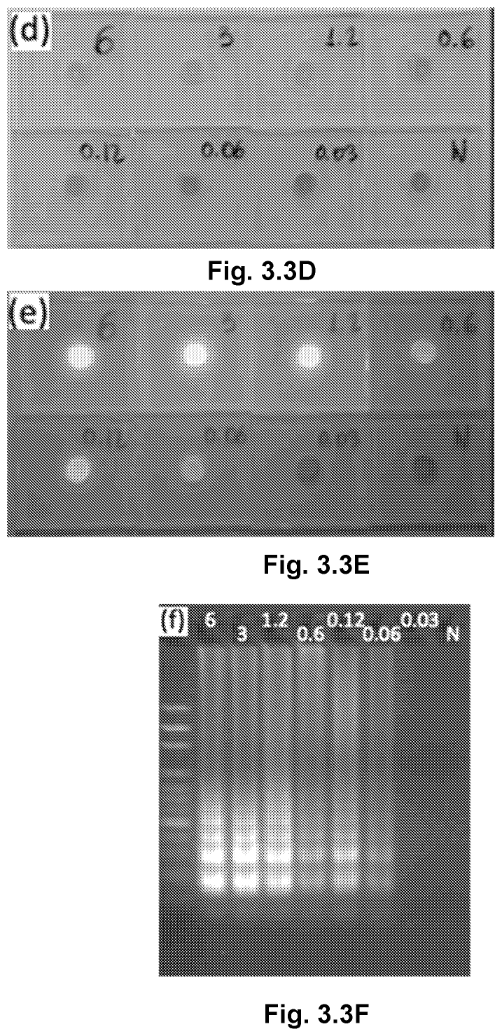

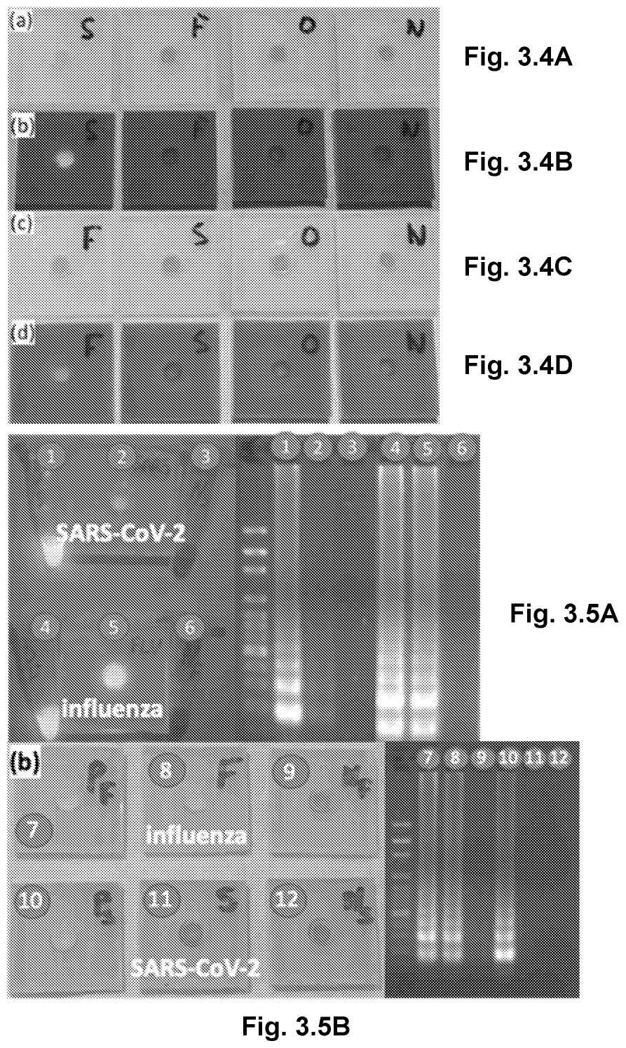

Device fabrication: The detection unit was made of a polycarbonate layer, a double-sided adhesive tape, two layers of thermoplastic films, and a paper pad as shown in FIG. 3.1A and FIGS. 3.1C-3.1E. The polycarbonate layer was shaped into a 2 cm×2 cm square from a 3-mm-thick polycarbonate sheet (McMaster-Carr, Elmhurst, IL) using a CNC milling machine (Sherline Products, Vista, CA), and a well of 4-mm diameter was created in the center. To create the laminated paper pad, one piece of Whatman™ 1 chromatography paper (Fisher Scientific) and two 75-μm-thick polyester thermal bonding lamination films (Lamination Plus, Kaysville, UT, USA) were cut into 3.5-mm-diameter circles using a Graphtec Craft Robo-S cutting plotter (Graphtec Corporation, Yokohama, Japan). The paper was then sandwiched between the two films and passed through a heated laminator, GBC® Catena 65 Roll Laminator (GBC, Lake Zurich, IL, USA), set at a rolling speed of “1” and at a temperature of 220° F. as previously descibed³¹. The laminated paper pad was attached to the polycarbonate container using double-sided adhesive tape (3M 9087 white bonding tape, R. S. Hughes, Sunnyvale, CA), forming the detection unit.

An exploded view of the 2-plex VLEAD is shown in FIG. 3.1A. The device consists of 3 components, including a buffer unit, a mixing unit, and 2 detection units. The buffer unit contains 4 wells in each side for the storage of a lysis buffer, a binding buffer, and 2 washing buffers for sample preparation. In each well (2) of the buffer unit one stainless steel ball (1) is placed at the bottom that functions as the valve as shown in FIG. 3.1B. The mixing unit has one well (5) in each side and it slides under the buffer unit through the sliding tracks (3) on both sides and in the middle. At the bottom of each mixing well, a protrusion (7) is created for inserting a detection unit. Gap (6) is for the balls that are protruded slightly; without this gap, the ball will be pushed up by the well edge during sliding. The detection unit contains a paper pad (11) that is laminated between two thermoplastic films (10) and attached to the polycarbonate well layer (8) using a double-sided adhesive tape (9). FIG. 3.1B shows a two-dimensional, cross-sectional view of the ball-based valve mechanism having corresponding parts to FIG. 3.1A. The valves are closed when the balls function as plugs for the buffer wells while protruding slightly at the bottom. The valves are actuated by the pins (4) in the mixing unit that lift the balls up when the pins are aligned with balls after sliding, releasing the reagents into the mixing unit.

A commercial 3D printer, Ultimaker 3 (Ultimaker, Geldermalsen, Netherlands), was used to fabricate the buffer unit and the mixing unit. The devices were printed using polylactic acid (PLA) with polyvinyl alcohol (PVA) as support material. The print layer height was set to 0.06 mm and the infill density was set to 100% for PLA and PVA. The ball valves used for each well were 4.0-mm-diameter corrosion-resistant 316 stainless steel balls (McMaster-Carr). To prevent accidental displacement or movement of the ball valves, a small amount of Akrowax™ 130 (Akrochem, Akron, OH, USA) was placed around the balls to melt and re-solidify, forming a breakable bond between balls and the buffer unit.

RT-LAMP reaction: Each 25-μL RT-LAMP mix contains 2.5 μL of 10× isothermal amplification buffer, 8 U Bst 2.0 WarmStart® DNA polymerase, 7.5 U WarmStart® RTx reverse transcriptase, 2.5 μL of 10× concentrated primer mix, and a final concentration of 1.4 mM deoxynucleotide triphosphate (dNTPs) and 6 mM MgSO4. The 25-μL volume was filled using nuclease-free water (not DEPC treated). Except for the nuclease-free water and dNTPs from ThermoFisher (MA, USA), all other reagents in the RT-LAMP mix were obtained from New England Biolabs (NEB, Ipswich, MA, USA). The 10× primer mix for SARS-CoV-2 contains 16 μM FIP/BIP, 2 μM F3/B3, and 8 μM LF/LB. The 10× primer mix for influenzaA H1N1 contains 16 μM FIP/BIP, 2 μM F3/B3, and 4 μM LF/LB. The primers were obtained from Integrated DNA Technologies (Coralville, Iowa, USA) and were chosen by following the literature^{32,33}.

In addition, 0.5 units of Antarctic thermolabile uracil-DNA glycosylase (UDG) and 0.7 mM of deoxyuridine triphosphate (dUTP) were added to the 25-μL RT-LAMP mix described above. UDG and dUTP were used to eliminate possible carryover contamination, reducing non-specific amplification and potential false-positives. UDG has been widely used to prevent carryover contamination without compromising sensitivity in LAMP and other nucleic acid amplification assays³⁴⁻³⁶. Initial work on the primer concentration comparison for SARS-CoV-2, the specificity tests, and the environmental sample experiments were carried out using the regular RT-LAMP mix. All other experiments were performed with UDG and dUTP added.

To achieve RT-LAMP without the need of connecting to a power outlet, we chose a commercially available, battery-powered coffee mug (Ember™ Travel Mug, Ember Technologies, Inc., Westlake Village, CA) as a heated water bath as we reported previously³¹. Prior to being placed in the Ember™ mug containing water at 62.5° C., the detection units were sealed using two pieces of tape (Fellows®) to cover the bottom and top parts. After 25 minutes of incubation, the detection units were taken out for colorimetric detection, which was carried out by adding 0.5 μL of 10,000× concentrate SYBR green I in dimethyl sulfoxide (ThermoFisher). We used SYBR green for endpoint detection of amplicons because its color change can be visualized by naked eye or recorded using a smartphone camera. To help visualization, a blue LED flashlight powered by one AA battery was used to observe the green fluorescence if target viruses were present. The amplified products can also be verified by gel electrophoresis. Note that RT-LAMP produces a mixture of different amplicon products; hence, it does not have one specific gel band as with RT-PCR³⁷.

Real-time RT-LAMP: We used a commercial real time thermal cycler to verify the incubation time required for SARS-CoV-2 and influenza A H1N1 virus assays. The real-time RT-LAMP experiments were carried out by adding 0.5 μL of 10× concentrate SYBR green I nucleic acid gel stain in dimethyl sulfoxide (ThermoFisher) to the 25 μL RT-LAMP reaction mix. The fluorescence signal from the reactions was read through the QuantStudio 3 real-time PCR system (ThermoFisher).

For both viruses, 4 concentrations were used along with no-template controls (NTC); and 3 replicates were carried out. For SARS-CoV-2 real-time assay, 10⁴, 10³, 10², and 10 genome equivalents (GEs) were spiked into 25-μL RT-LAMP reactions. For influenza A H1N1 virus assay, RNA of viruses in the amount of 600, 60, 6, and 1.2 TCID₅₀ were spiked into 25-μL RT-LAMP reactions.

For experiments to study the effects of primer concentrations we used SARS-CoV-2 RNA of 3 RNA amounts (10⁴, 10³, and 10² GEs) and compared the effects of two different primer concentrations. The RT-LAMP mixtures were incubated for 45 min to analyze which primer conditions would give faster amplification without producing non-specific amplification. For the endpoint colorimetric detection in we compared RT-LAMP results after 20, 25, and 30 min of incubation at 62.5° C. Three sets of repeat experiments were carried out for each primer concentration. The same SARS-CoV-2 RNA concentration was used for all the positive controls (10² GEs).

Assay sensitivity and specificity: To assess the sensitivity of the RT-LAMP assay for detection of SARS-CoV-2, RNA was extracted from a stock of SARS-CoV-2/human/USA/UF-1/2020 (GenBank accession no. MT295464). The genome equivalents per microliter (GEs/μL) of the extracted RNA was estimated from a standard curve based on a RT-PCR assay³⁸ and corresponded to approximately to 1×10⁶ GEs/μL. 10-fold serial dilutions were made using RNA storage solution (Invitrogen), and 1 μL of purified RNA of the different concentrations used in 25-μL RT-LAMP reactions, along with an NTC.

For influenza A H1N1 virus, RNA was extracted and purified using the Zymo Viral Magbead kit (Zymo Research) from a stock of influenza virus H1N1 strain A/Mexico/4108/2009 that was at a titer of 6×10⁶ TCID₅₀/mL. Again, 10-fold serial dilutions were made using RNA storage solution, and 1 μL of purified RNA at different concentrations was added into 25-μL RT-LAMP reactions, along with an NTC. For more accurate results, different dilutions were made between 6 and 0.03 TCID₅₀/μL.

We determined the detection specificities of SARS-CoV-2 and influenza A H1N1 virus assays by carrying out the following experiments: (a) we used the primer set for SARS-CoV-2 detection to test the genomic RNAs of SARS-CoV-2, influenzaA H1N1, and CoV-OC43, respectively, to confirm it would not produce non-specific amplification for the latter two, and (b) we used the primer set for influenza A H1N1 virus detection to test these three virus RNAs. For both experiments, 1 μL of each virus RNA sample was added into 25-μL RT-LAMP reactions, along with an NTC.

Multiplexed detection: The 2-plex VLEAD operation starts with adding a 140 μL sample into each mixing well, which is the recommended volume used in the commercial QIAamp Viral RNA mini kit (QIAGEN). Immediately after, the mixing unit slides to the first reservoirs of the buffer unit, which have been pre-loaded with 560 μL of lysis buffer (AVL, QIAGEN), discharging them into the mixing unit to mix with each sample. To keep the 1:4:4 volume ratio of sample:(lysis buffer):(binding buffer) as in the kit protocol, 560 μL of ethanol have been pre-loaded into the second reservoirs as the binding buffer. After the lysis buffer flows down, the mixing unit is moved to the second reservoirs to discharge the binding buffer, which promotes RNA to be absorbed onto the paper pad as the solution goes through the detection units. After these solutions go completely through the paper, the mixing unit is slid again one at a time to the third and fourth reservoirs, which are pre-loaded with 1 mL of AW1 and AW2 (QIAGEN) wash buffer, respectively. Finally, the detection units are removed from the mixing unit, followed by adding the 25-μL RT-LAMP mix into each unit, and incubating them in the coffee mug at 62.5° C. for 25 min.

For heat inactivated SARS-CoV-2 samples, and in real world situations, 140 μL samples are added to the mixing wells as described above, followed by the discharge of the lysis buffer through the ball valve. However, as a safety precaution for our experiments, the influenza A H1N1 virus sample was mixed with AVL at a ratio of 1:4 in a biosafety level 2+ laboratory prior to processing the sample in the device. Thus, in this case, the lysed sample would be added to the mixing unit followed by the discharge of the binding buffer, using only 3 of the 4 wells in the buffer unit.

Environmental samples: Environmental samples were collected between February and March of 2020 by swabbing a handle of the main entry door of a building at the University of Florida, as described previously³⁹. The environmental samples tested in this work were enumerated as samples #1, #2, #3, #4, and #5 corresponding to their collection on February 19, February 20, February 21, March 2, and March 4, respectively³⁹. RNA was purified from the samples using a QIAamp Viral RNA Mini Kit, and the purified RNA stored at −80° C. in the presence of SUPERase-in RNase inhibitor (ThermoFisher). For our experiments, samples were created by mixing 3.5 μL of purified RNA of the 5 different environmental samples with 14 μL of AVL buffer and 14 μL of ethanol in a biosafety hood. The 31.5-μL mix was loaded into the detection unit using a pipette. After the sample mix had completely gone through the paper pad, 100 μL of AW1 was passed through the unit, followed by 100 μL of AW2. After RNA enrichment and purification, the detection units went through RT-LAMP amplification as described above.

Controls and samples were run in parallel for both viruses, SARS-CoV-2 and influenza A H1N1 viruses. Their preparation was in the following order: negative controls, samples, and positive controls. This order was chosen to reduce the opportunities of possible contamination during sample handling, leading to possible false positives. For the negative controls, nuclease-free water was added. For positive controls, 1 μL of purified RNA of each virus was added to the respective RT-LAMP mix.

### Results and Discussion

Device design and fabrication: FIG. 3.1A shows the design of 2-plex VLEAD for the simultaneous detection of SARS-CoV-2 and influenza A viruses. The device consists of a buffer unit in the top, a mixing unit in the middle, and two detection units at the bottom. We borrowed the concept of our previously reported singleplex VLEAD device for Zika virus detection³¹. In addition to the difference in the multiplexing capability between singleplex and duplex devices, the 2-plex device has another sliding mechanism in the middle for extra support. Since the 2-plex device simultaneously processes two samples in the same sequence, both detection units will be ready for the subsequent step.

The device integrates all the necessary steps for NAAT, including virus lysis, RNA enrichment and purification, amplification, and detection. Fluid-control valves are employed to perform sample preparation by sequential release of the reagents from the buffer unit into the mixing unit without the need of basic laboratory equipment. The valves consist of stainless-steel balls placed at the bottom of each buffer well to prevent the reagents from flowing down until desired. These ball-based valves protrude 1.5 mm from the bottom of the buffer unit so that the pins in the mixing unit, designed to be at the same level as the bottom surface of the buffer unit, lift the balls up allowing the reagents to flow down when the pins are aligned with the balls, as shown in FIG. 3.1B. To prevent balls from lifting up while sliding the mixing unit before the pins are aligned with the balls, two gaps are created as clearance at the top of each mixing well to let the balls pass. Additionally, to prevent ball displacement and fluid leakage during the device transportation, a breakable bond is created between the ball and the reservoir using biocompatible wax. First, a small piece of wax is placed around each ball valve, followed by heating the device to melt the wax. The wax is then cooled down to re-solidify around the ball and create a bond to prevent any undesired ball movement. The bond is breakable later on when a pin lifts up the ball.

The RNA enrichment process is driven by the capillary forces generated by the chromatography paper in the detection units, eliminating the need of external equipment. An untreated cellulose chromatography paper was chosen for RNA enrichment because it has shown better results for RNA enrichment of influenza viruses when compared with glass-fiber paper and FTA card¹⁷. Additionally, the device does not require an elution step as in the QIAGEN kit, where the purified RNA needs to be eluted before amplification, which has a significant disadvantage: (1) the purified RNA is diluted during elution and (2) not all the RNA on the column can be eluted. Involving no RNA transferring between tubes as in the lab operation, this device avoids possible contamination and degradation issues.

Compared to other multiplexed POC platforms developed for simultaneous detection of SARS-CoV-2 and influenza viruses, our system offers higher sensitivity and specificity than those antigen tests²⁹ due to amplification and genetic identification, and processing larger sample volume (140 μL in our device) than typical microfluidic platforms such as the centrifugal RT-PCR microfluidic devices that can process a few μL of samples³⁰. The ability of processing a larger sample volume can lead to lower limit of detection since more virus RNA is enriched onto the paper pad of the detection unit.

RT-LAMP reaction time: We determined the time needed for optimal RT-LAMP detection of the target virus using the QuantStudio-3 real-time amplification system. For SARS-CoV-2, all samples containing various concentrations of RNA reached a plateau within 25 min. as shown in FIG. 3.2A. The reactions were incubated for 35 min., and no non-specific amplification was observed in the NTC. These results were confirmed by that 25-min RT-LAMP reaction time was sufficient to detect SARS-CoV-2 RNA in our device. This 25-min amplification time is significantly shorter than the conventional RT-PCR used for COVID-19 diagnosis^{40,41}. Note that RT-LAMP involves many complicated reaction steps, and the resulting fluorescence signal does not necessarily correlate with the starting viral load. However, the threshold time measured by the instrument can be used to correlate with the viral load as in RT-PCR⁴². FIG. 3.2B shows the calibration curve, indicating the feasibility of semi-quantitative SARS-CoV-2 detection.

FIG. 3.2A shows real-time RT-LAMP amplification for SARS-CoV-2 showing fluorescent signal of 10⁴, 10³, 10², and 10 genome equivalents (GEs) as a function of reaction time. NTC, no-template control. The average of three replicates was used. FIG. 3.2B shows the calibration curve between the threshold time (Ct) and SARS-CoV-2 GEs in each reaction (in log scale). The error bars indicate one standard deviation, generated from 3 replicates of each concentration of SARS-CoV-2 RNA samples. FIG. 3.2C shows real-time RT-LAMP amplification for influenza A virus, showing fluorescent signal of 600, 60, 6, and 1.2 Median Tissue Culture Infectious Dose (TCID50) of influenza A virus as a function of RT-LAMP time. NTC, no-template control. The average of three replicates was used. FIG. 3.2D shows the calibration curve between the threshold time (Ct) and influenza A virus TCID50 in each reaction (in log scale). The error bars indicate one standard deviation, generated from 3 replicates of each concentration of influenza A virus RNA samples.

We used H1N1 pdm2009 as an example of influenza A virus for this work, and their real-time RT-LAMP assay results are shown in FIG. 3.2C. All samples in different concentrations reached a plateau within 20 min. The reactions were incubated for 35 min, and no non-specific amplification was observed in NTC. The results suggest that 20-min incubation was sufficient for detecting influenza A viruses in our device. However, we used 25-min incubation for simultaneous detection of SARS-CoV-2. FIG. 3.2D shows the calibration curve between the threshold time (Ct) and influenza A virus amount, indicating that semi-quantitative detection is feasible as shown previously¹⁷. Note that threshold time was automatically reported from the real-time PCR machine.

Effects of primer concentrations: For the detection of SARS-CoV-2 genomic RNA, we chose the RT-LAMP primers developed by Baek et al.³² However, the primer concentrations used by Baek et al.³² (5 μM for external primers F3 and B3, 20 μM for internal primers FIP and BIP, and 5 μM for loop primers LF and LB) are different from what we previously reported (2 μM for F3/B3, 16 μM for FIP/BIP, and 8 μM for LF/LB)³¹, which were based on recommendations by the RT-LAMP reagent manufacturer, NEB.

Using the NEB primer concentrations reported previously³¹, a color change in the reaction tube was observed after 20 min. of RT-LAMP for a sample containing 100 GEs virus RNA. The color change was more pronounced after 25 min. of RT-LAMP in another tube (note that separate tubes must be used for each condition because the colorimetric detection was carried out at the end of RT-LAMP). In contrast, no color change was observed after 20 min. of RT-LAMP, and the color change was observed only after 25 min. of RT-LAMP when we employed the primer concentrations used by Baek et al.³² These results were confirmed using real-time RT-LAMP assay, in which the signals were observed a bit later under the conditions used by Baek et al. than under the NEB conditions for all three concentrations of SARS-CoV-2 viruses. This difference in RT-LAMP time between two experimental conditions was larger for 10² GEs virus RNA than 10³ and 10⁴ GEs virus RNA. It should be noted that good specificity was attained using both approaches, with no non-specific amplification after 45 min. of RT-LAMP when no template was present. On the other hand, the condition used by Baek et al showed better linear correlation between the number of GEs and the threshold time than the NEB conditions. For the following experiments, we chose to employ the NEB conditions.

Assay sensitivity and specificity: The RT-LAMP assay for SARS-CoV-2 detection showed high sensitivity and a limit of detection (LoD) of 2 GEs. We first studied the assay using 100, 10, and 1 GEs of SARS-CoV-2 (as shown in FIGS. 3.3A and 3.3B) and observed the positive signals in all 3 replicates of 100 and 10 GEs. For 1 GE samples, we observed only 2 out of 3 replicates, indicating the LoD of our assay is at least at 10 GEs. The results were confirmed using gel electrophoresis (FIG. 3.3C). To further determine the LoD between 1 and 10 GEs, we carried out the second experiment using 10, 5, 2, and 1 GEs of SARS-CoV-2, and observed positive signals in all 3 replicates of 10, 5, and 2 GEs. Similarly, we observed positive signals only 2 out of 3 replicates for 1 GE samples. These results are summarized in Table 1. The capability of detecting 2 GEs of SARS-CoV-2 shows that the device described herein has comparable limit of detection to, if not better than, the gold-standard RT-PCR assay developed by CDC (which is 5 copies/reaction^{40,43}), as well as other RT-LAMP assays for SARS-CoV-2 detection in the literature^{21,44} and can be used at the point of collection, rather than in a lab.

FIG. 3.3A provides pictures of the detection units taken under room light after RT-LAMP assay at 62.5° C. for 25 min. Amount of SARS-CoV-2 RNA are marked on the devices, 100, 10, and 1 GEs, as well as a negative control (N). FIG. 3.3B shows the same devices of 3.3A under blue LED. FIG. 3.3C shows gel electrophoresis of those samples in 3.3A. The left lane is for DNA ladder while other lanes are marked at the top. FIG. 3.3D shows pictures of the detection units taken under room light after RT-LAMP assay at 62.5° C. for 25 min. Amount of influenza A virus RNA are indicated, 6, 3, 1.2, 0.6, 0.12, 0.06, and 0.03 TCID₅₀, as well as a negative control (N). FIG. 3.3E shows the same devices of 3.3D under blue LED. FIG. 3.3F shows gel electrophoresis of those samples in 3.3D. The left lane is for DNA ladder while other lanes are marked at the top.

TABLE 1
RT-LAMP assay results for SARS-CoV-2 detection

| RNA | | | |
|---|---|---|---|
| amount | First | Second | |
| (GEs) | Experiment* | Experiment* | Total* |
| 100 | 3/3 | | 3/3 |
| 10 | 3/3 | 3/3 | 6/6 |
| 5 | | 3/3 | 3/3 |
| 2 | | 3/3 | 3/3 |
| 1 | 2/3 | 2/3 | 4/6 |
| NTC | 0/3 | 0/3 | 0/6 |

Note:
*the results are listed as the number of positive results/the number of experiments.

Similarly, the assay for influenza A virus showed a LoD of 0.06 TCID₅₀, which is approximately 6 GEs (based on about 100 GEs per virion and 1 virion corresponds to 1 TCID₅₀ unit). We studied the RT-LAMP assay using concentrations from 6 TCID₅₀ to 0.003 TCID₅₀ of influenza A viruses and observed the positive signals in all 5 replicates of 0.06 TCID₅₀ and higher, as shown in FIGS. 3.3D and 3.3E. However, for samples containing less than 0.06 TCID₅₀ we did not observed signals in any replicates. The results were also confirmed by gel electrophoresis (FIG. 3.3F). This LoD of 6 GEs is at least similar, if not better, when compared to other RT-LAMP assays for detection of influenza viruses at the POC, which is in the range of 10 to 100 copies per reaction^{15,16}. Overall, RT-LAMP assays for both SARS-CoV-2 and influenza A viruses have a LoD of <10 GEs per reaction, which is comparable to the gold standard RT-PCR assays.

FIGS. 3.4A-3.4B shows both assays for SARS-CoV-2 and influenza A viruses have good specificity in our devices. The SARS-CoV-2 assay showed no cross-reactivity with influenza virus A and human coronavirus OC43 (CoV-OC43) as shown in FIGS. 3.4A and 3.4B. CoV-OC43 is a coronavirus that is known to infect humans and cause the common cold. Similarly, the influenza A virus assay showed no cross-reactivity with SARS-CoV-2 and CoV-OC43 as shown in FIGS. 3.4C and 3.4D. These experimental results are expected because the SARS-CoV-2 primers had been previously tested against a panel of RNA samples of related coronaviruses, a panel of human and avian influenza viruses, and a panel of other respiratory disease-causing viruses³². The RT-LAMP primers for influenzaA H1N1 viruses had also been tested previously against other subtypes of influenza A viruses, though they have not been tested against any coronaviruses³³.

Device testing using control samples: Using the device in FIG. 3.1A, we were able to detect SARS-CoV-2 and influenza A viruses using inactivated virus samples (FIG. 3.5A). We obtained heat-inactivated SARS-CoV-2 samples from BEI Resources (USA-WA1/2020, NR-52286), which were deposited by CDC. The influenzaA H1N1 pdm2009 was produced by the laboratory of Dr. John Lednicky as reported previously⁴⁵ and the samples in cell culture media were lysed with AVL buffer in biosafety level 2+ environment to meet safety requirements. The total time for sample preparation to flow the respective solutions through the device and to enrich RNA onto the paper pad in the detection unit was about 25 min.

FIG. 3.5A shows simultaneous detection of heat-inactivated SARS-CoV-2 and influenzaA H1N1 viruses using the 2-plex VLEAD device. Pictures of (1) positive control in a tube for SARS-CoV-2 assay, (2) detection unit after processing SARS-CoV-2 sample, (3) negative control in a tube for SARS-CoV-2 assay, (4) positive control in a tube for influenza A virus assay, (5) detection unit after processing influenza A H1N1 virus sample, and (6) negative control in a tube for influenza A virus assay are on the left. Pictures were taken under blue LED. Gel electrophoresis of these samples are shown on the right, in which the left lane is DNA ladder while other lanes are marked at the top. FIG. 3.5B shows multiplexed detection of SARS-CoV-2 and influenza A H1N1 viruses in environmental sample #2. Pictures of detection units for influenza A H1N1 virus assay (7) positive control, (8) sample #2, (9) negative control, and for SARS-CoV-2 assay (10) positive control, (11) sample #2, (12) negative control are on the left. Gel electrophoresis of these samples are shown on the right, in which the left lane is DNA ladder while other lanes are marked at the top.

Multiplexed detection using environmental samples: We tested 5 environmental samples that were spared from another study⁴⁵. These samples were identified with numbers only and blinded to the researchers who performed 2-plex assays. The results were then provided back to the investigator of the previous study and compared with those results using RT-PCR³⁹. The comparison show that we were able to detect both viruses, SARS-CoV-2 and influenza A H1N1 in sample #3, which is in agreement with the results obtained by RT-PCR and sequencing³⁹. FIG. 3.5B shows the detection results of sample #2 that contains influenza A virus only. All results for 5 environmental samples are summarized in Table 2. Since the samples were the leftovers from the previous study³⁹, some of them are insufficient for replicate experiments. Overall, however, we demonstrated the decent specificity and sensitivity of our assay, with only 1 false positive and 1 false negative among a total of 19 experiments. The results correspond to a 90.0% (9/10) of positive percent agreement (PPA), an 88.9% (8/9) of negative percent agreement (NPA), and an overall percent agreement of 89.5% (17/19).

TABLE 2
RT-LAMP assay for environmental samples
and its comparison with RT-PCR

| | SARS-CoV-2 | | Influenza A H1N1 | |
|---|---|---|---|---|
| Sample # | detected* | RT-PCR | detected* | RT-PCR |
| 1 | 0/1 | no | 1/1 | yes |
| 2 | 0/2 | no | 2/2 | yes |
| 3 | 2/2 | yes | 2/2 | yes |
| 4 | 0/2 | no | 2/3 | yes |
| 5 | 0/2 | no | 1/2 | no |

Note:
*the results are listed as the number of positive results/the number of experiments; “yes” or “no” indicates whether SARS-CoV-2 or Influenza A H1N1 virus was present or absent in the corresponding samples based on RT-PCR.

Conclusions: We have developed a rapid and sensitive multiplexed POC testing platform, 2-plex VLEAD, for simultaneous detection of SARS-CoV-2 and influenza A H1N1 viruses in 50 min. (˜25 min. for sample preparation and ˜25 min. for RT-LAMP assay). To our knowledge, our 2-plex VLEAD is the first POC platform that can simultaneously detect both viruses using NAAT integrating all the necessary steps including sample preparation, RNA amplification, and detection into a single platform without the need of bulky or sophisticated laboratory equipment. Other platforms have been reported for POC detection using NAAT for SARS-CoV-2^{21,46} or influenza viruses^{15,18,47}, for laboratory setting using NAAT for both viruses^{48,49}, and for healthcare setting detection approved by FDA for emergency used authorization⁵⁰. Nevertheless, they are not for multiplexed detection of SARS-CoV-2 and other respiratory viruses such as influenza virus at POC. A multiplexed POC platform will be useful for testing individuals suspected of either SARS-CoV-2 or influenza infections, especially during flu seasons.

The 2-plex VLEAD can be adapted for testing different variants of SARS-CoV-2, subtypes of influenza viruses (e.g., A and B), or other types of viruses, depending on the need and potential outbreaks. The reagents can be pre-packaged in the buffer unit for storage and transportation for testing at the POC and the ball-based valves have shown no leakage for several weeks when wax is used to fix the ball to the respective buffer well. Therefore, the platform has the potential to help reduce disease transmission by bringing the tests close to patients or other sites in need.

### Example 4

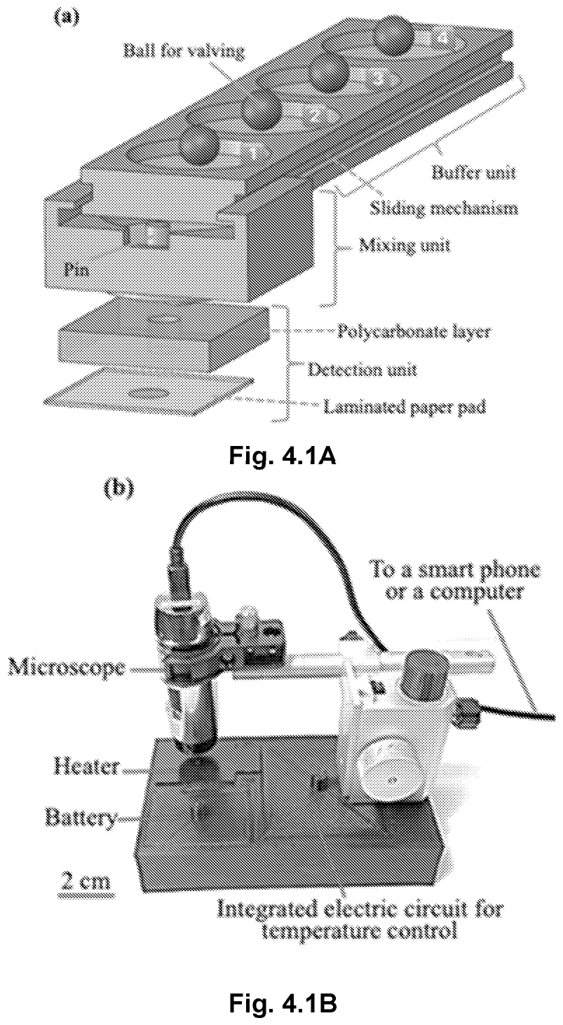

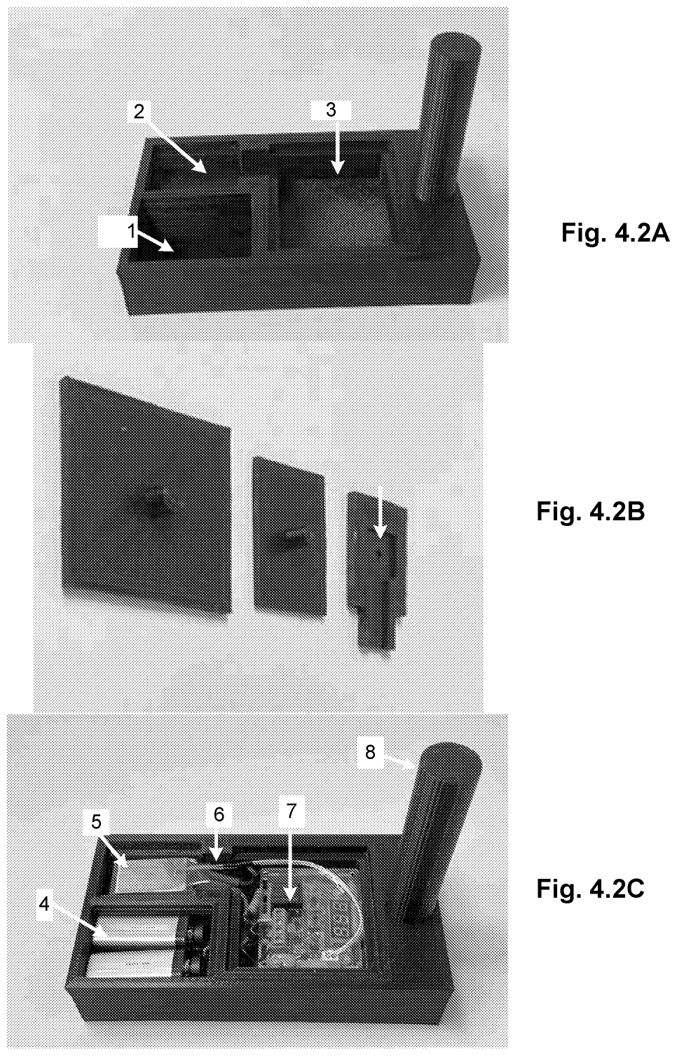

The end-point detection described above (FIGS. 3.1A-3.5B) only provides binary results (presence and absence of a virus). In some circumstances (such as detection of MAYV, SARS-CoV-2, or other viruses), quantitative information is desirable. We have developed a real-time detection platform (also called a portable analysis device, above) that offers quantitative information. As shown in FIGS. 4.1B, this platform consists of a pencil-shaped digital microscope and 3D-printed stand with a base containing 3 chambers for a heater, battery, and integrated electronic circuit. The detection unit in FIG. 4.1A is placed in the seat designed in the heater chamber's cap. The digital microscope was programed to acquire fluorescence images at a frequency of every minute. The LED right was “on” for 5 s and “off” for 55 s to avoid possible photobleaching. Two Python-based App, compatible with computer and cellphone operating systems, were developed for measuring fluorescence intensity and plotting the data in real time. The total weight of the detection platform is 983 g; thus, it can be handheld and portable. FIG. 4.1A provides an exploded view of the VLEAD consisting of a buffer unit, a mixing unit, and a detection unit. The buffer unit comprises 4 reservoirs for the storage of a lysis buffer (reservoir 1), a binding buffer (reservoir 2), and 2 washing buffers (reservoirs 3 and 4). In each funnel-shaped reservoir, one stainless-steel ball is placed at the bottom that functions as a valve (the balls are above the reservoirs for visualization). The mixing unit contains a well and a pin, and it slides along the buffer unit through the sliding tracks on both sides of the buffer unit. When the pin in the mixing unit is aligned with the ball, it lifts the ball to open the valve and allow the solution to flow down through the mixing unit and into the detection unit. The paper pad in the detection unit (2 cm×2 cm) is used to collect RNA for subsequent RT-LAMP application. FIG. 4.1B is an illustration of an assembled real-time detection platform consisting of a pencil-shaped microscope and 3D-printed chambers for a heater, battery, and integrated electrical circuit.

FIGS. 4.2A-4.2C provide photographs of the components for the portable analysis device shown in FIG. 4.1B. The device was 3D printed in black polylactic acid (PLA), although other suitable materials or production methods can be envisioned by one of ordinary skill in the art. FIG. 4.2A shows the chambers for the battery (1), heater and detection unit (2), and temperature controller (3). FIG. 4.2B shows (left to right) caps for the temperature controller chamber, the battery chamber, and the heater chamber with a seat for the detection unit (marked by arrow). FIG. 4.2C shows the assembled portable analysis device for real-time detection platform without the caps. Batteries (4) are in the battery chamber, a heater (5) and sensor (6) are installed in the heater and detection unit chamber, and the heater/sensor are connected to the temperature controller (7) in the temperature controller chamber. Microscope holder (8) serves as a support for a microscope.

In an embodiment, the real-time detection platform is composed of a portable stand, digital microscope (e.g., AM4117MT-G2FBW, Dino-Ute, USA), a temperature controller, a heater, and two 9-volt chargeable batteries. The stand was 3D printed from polylactic acid (PLA), with a base containing three chambers for the heater, batteries, and temperature controller. Three caps were also 3D-printed for covering the three chambers. The cap for the heater chamber was designed to contain a 2×2 cm² slot, which properly fit the detection unit. In addition, two 5-mm holes were created in the cap, enabling real time detection of the RT-LAMP reactions (one for the sample and the other for the negative control).

The digital microscope possesses two bandpass filters which allows only desired excitation and emission wavelength to be transmitted through. The peak excitation wavelength is 465 nm while the emission band is 510-545 nm. These wavelengths are compatible with the excitation/emission wavelengths of SYTO9, which was used as fluorescent dye in our real-time detection platform. We replaced SYBR Green with SYTO9 since SYTO9 has far less inhibitory effects on RT-LAMP than SYBR Green (48). Our study showed that 4 μM of SYTO9 is optimal for obtaining highest fluorescence intensity without inhibition on RT-LAMP.

The heater we used for RT-LAMP is a positive temperature coefficient (PTC) heater (Bolsen Tech, USA), which can be powered by batteries, a laptop, or a cellphone. A commercially available, low-cost temperature controller was used to switch the PTC heater on or off. The controller's thermostat sensor was attached to the PTC heater and the sensor temperature was calibrated to set the RT-LAMP reaction at nearly 62.5° C. We characterized the heating system by placing a T-type thermocouple in the RT-LAMP well. The results illustrate that the RT-LAMP temperature was maintained between 62.5-63.1° C., which is within the standard temperature range (60-65° C.) recommended for LAMP.

### ASPECTS OF THE DISCLOSURE

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. A multiplex device for preparing a sample for nucleic acid detection comprising at least two parallel buffer units comprising a plurality of buffer wells arranged in a row, wherein each buffer well comprises a ball valve in a bottom of the well; a mixing unit associated with each buffer unit, wherein the mixing unit comprises a mixing well and a pin, wherein the mixing unit is slidably connected to a bottom of the buffer unit such that the mixing unit can be moved along a length of the buffer unit to align with each buffer well in turn, wherein when the mixing unit is aligned with a buffer well the pin engages with the ball valve to release buffer from the buffer well into the mixing well; and a detection unit removably coupled to the bottom of each mixing unit to receive fluids from the mixing well; wherein the parallel buffer units are connected to form a multiplex device.

Aspect 2. The device of aspect 1, further comprising an amplification unit coupled to an end of each buffer unit, the amplification unit comprising a buffer well comprising a ball valve in the bottom of the well, wherein the mixing unit is slidably connected to a bottom of the amplification unit such that the mixing unit can be moved along a length of the amplification unit after the buffer unit to align with an amplification well, wherein when the mixing unit is aligned with the amplification well the pin engages with the ball valve to release buffer from the amplification well into the mixing well; and wherein the amplification unit is selected from a LAMP unit for processing DNA or an RT-LAMP unit for processing RNA.

Aspect 3. The device of aspects 1 or 2, further comprising a drain unit removably coupled to the bottom of each detection unit to receive fluids from the detection unit.

Aspect 4. The device of any of aspects 1-3, wherein the detection unit comprises an absorbent layer, wherein the absorbent layer is selected from chromatography paper, cellulose paper, a membrane, and glass microfiber paper.

Aspect 5. The device of any of aspects 1-4, wherein the buffer units are modular and are removably connected to each other along a side of the buffer unit.

Aspect 6. The device of any of aspects 1-4, wherein the buffer units are permanently connected to each other.

Aspect 7. The device of any of aspects 1-7, wherein the device is comprised of 2 to 10 buffer units.

Aspect 8. The device of aspects 5 or 6, wherein a first well of each buffer unit can receive a sample, and wherein the sample can be the same for each buffer unit or not the same for each buffer unit.

Aspect 9. The device of any of aspects 3-8, wherein the drain unit comprises a tube, such that buffer can be extracted from the drain unit by a syringe or vacuum.

Aspect 10. The device of any of aspects 2-9, wherein each buffer unit comprises four buffer wells and each LAMP unit comprises a single well.

Aspect 11. The device of any of aspects 1-10, further comprising a heating unit, wherein the heating unit comprises a seat for the detection unit, wherein the heating unit provides a temperature of 57° C. to 67° C.

Aspect 12. The device of aspect 11, wherein the heating unit further comprises at least one cartridge heater.

Aspect 13. The device of aspect 11, wherein the heating unit is powered by a portable electronic device or batteries.

Aspect 14. The device of any of aspects 11-13, wherein the heating unit is a component of a portable analysis device, the analysis device further comprising a battery, a microscope, and a temperature control circuit, wherein the heating unit has a cap that provides a seat for the detection unit.

Aspect 15. The device of aspect 14, further comprising an imaging apparatus, wherein the imaging apparatus detects a signal of nucleic acid amplification in the detection unit in real time.

Aspect 16. The device of any of aspects 1-15, further comprising a sample collection unit, wherein the sample collection unit comprises a sample well having a ball valve in a bottom of the sample well, wherein when a first buffer well receives a bottom of the sample collection unit the ball valve of the collection unit is engaged to release contents of the sample collection unit into the buffer well.

Aspect 17. A multiplex device for detecting nucleic acid from a nucleic acid source comprising: at least two parallel buffer units, wherein each buffer unit comprises a plurality of buffer wells arranged in a row, wherein each buffer well comprises a ball valve in a bottom of the well; wherein a nucleic acid source and a first buffer is provided to a first buffer well, a second buffer is provided to a second buffer well, a third buffer is provided to a third buffer well, and a fourth buffer is provided to a fourth buffer well; an amplification unit coupled to an end of each buffer unit, the amplification unit comprising a buffer well, wherein the coupling forms a singleplex unit; wherein if the nucleic acid source is DNA the amplification unit is a LAMP unit and if the nucleic acid source is RNA the amplification unit is an RT-LAMP unit; wherein an amplification buffer is provided to an amplification buffer well; a mixing unit associated with each singleplex unit, wherein the mixing unit comprises a mixing well and a pin, wherein the mixing unit is slidably connected to a bottom of the singleplex unit such that the mixing unit can be moved along a length of the singleplex unit to align with each buffer well in turn beginning with the first buffer well and ending with the amplification unit, wherein when the mixing unit is aligned with a buffer well the pin engages with the ball valve to release buffer from the buffer well into the mixing well such that nucleic acid is produced in the mixing unit from the nucleic acid source after the mixing unit engages with the amplification unit; a detection unit removably coupled to the bottom of each mixing unit to receive buffer and nucleic acid from the mixing well, wherein the nucleic acid is collected and amplified in the detection unit; and a drain unit removably coupled to the bottom of each detection unit to extract buffer from the detection unit; and a heating unit to amplify the nucleic acid product wherein the heating unit comprises a seat for the detection unit, wherein the heating unit provides a temperature of 57° C. to 67° C.

Aspect 18. The device of aspect 17, wherein the detection unit can be removed from the buffer unit and analyzed to determine whether liquid received by the detection unit contains nucleic acid from a target virus or microorganism.

Aspect 19. The device of aspect 18, wherein each detection unit can be configured to detect the same target virus or microorganism or a different target virus or microorganism.

Aspect 20. The device of aspects 18 or 19, wherein the target virus is selected from the group comprising SARS-CoV-2 virus, influenza virus, Zika virus, Dengue virus, Chikungunya virus, Ebola virus, hepatitis A virus, hepatitis B virus, hepatitis C virus, human immunodeficiency viruses (HIV) and Mayaro virus.

Aspect 21. The device of any of aspects 18-20, wherein the target microorganism is selected from the group comprising Escherichia coli (E. coli), enterococci, Salmonellae, Streptococcus pneumoniae, Staphylococcus aureus and Pseudomonas aeruginosa.

Aspect 22. The device of aspect 19, wherein the device comprises two singleplex units such that the device can detect both SARS-CoV-2 virus and influenza virus.

Aspect 23. The device of any of aspects 17-22, wherein the heating unit is a component of a portable analysis device, the analysis device further comprising a battery, an imaging apparatus, and a temperature control circuit.

Aspect 24. The device of aspect 23, wherein the imaging apparatus detects a signal of nucleic acid amplification in the detection unit in real time.

Aspect 25. The device of aspect 24, wherein the analysis device provides viral load information.

Aspect 26. A method for preparing a sample to be used for detecting nucleic acid from a nucleic acid source comprising: placing a first liquid sample in at least a first well of a first buffer unit of a nucleic acid detecting apparatus; placing at least a second, third and fourth liquid in at least second, third and fourth wells, respectively, of the first buffer unit of the nucleic acid detecting apparatus; placing a second liquid sample in at least a first well of a second buffer unit of the nucleic acid detecting apparatus; placing at least a second, third and fourth liquid in at least second, third and fourth wells, respectively, of the second buffer unit of the nucleic acid detecting apparatus; wherein a mixing unit is slidably coupled to a bottom of each buffer unit allowing a user to create relative movement between the mixing unit and the buffer unit by exerting a manual force on at least one of the buffer unit and the mixing unit; and exerting the manual force on at least one of the buffer unit and the mixing unit to slide the mixing unit along the buffer unit to cause the first, second, third and fourth wells to come into temporary alignment with the mixing unit for respective time periods; wherein a valve mechanism in each of the wells causes the first, second, third and fourth liquids to be released from the first, second, third and fourth wells, respectively, into the mixing unit, in turn, when the manual force is exerted to cause the second, third and fourth wells to come into temporary alignment with the mixing unit for respective time periods such that each liquid sample is prepared for nucleic acid amplification as it combines with the buffers from the wells in the mixing unit; and wherein a detection unit is coupled to a bottom of the mixing unit and receives fluid from the mixing unit.

Aspect 27. The method of aspect 26, wherein the apparatus further comprises an RT-LAMP unit coupled to an end of each buffer unit after the fourth well, the RT-LAMP unit comprising a well and a valve mechanism.

Aspect 28. The method of aspect 27, further comprising wherein the first well of the first buffer unit contains a lysis buffer, wherein the second well of the first buffer unit contains a binding buffer, wherein the third and fourth wells of the first buffer unit contains a washing buffer, and wherein the RT-LAMP unit contains an RT-LAMP buffer specific to a first target nucleic acid sample; and wherein the first well of the second buffer unit contains a lysis buffer, wherein the second well of the second buffer unit contains a binding buffer, wherein the third and fourth wells of the second buffer unit contains a washing buffer, and wherein the RT-LAMP unit contains an RT-LAMP buffer specific to a second target nucleic acid sample; wherein as the mixing unit for a respective buffer unit is moved from well to well, cells in each liquid sample are lysed by the lysing buffer to release nucleic acid, the released nucleic acid in the sample is bound by the binding buffer, and the nucleic acid is purified by the washing buffers and collected on a detection pad in the detection unit.

Aspect 29. The method of any of aspects 26-28, wherein the first liquid sample and the second liquid sample are from a same source, and wherein the first target nucleic acid sample and the second target nucleic acid sample are different.

Aspect 30. The method of aspect 28, further comprising amplifying the nucleic acid by sliding the mixing unit from alignment with the fourth well to the RT-LAMP unit to release buffer from the RT-LAMP unit into the mixing unit, wherein RNA is amplified on the detection pad.

Aspect 31. The method of aspect 30, further comprising draining the collected buffer from the detection unit via a drain unit coupled to a bottom of the detection unit.

Aspect 32. The method of aspect 30, further comprising removing each detection unit from the apparatus and analyzing each detection unit to determine whether the liquid received by the detection unit contains target nucleic acid from a target virus or microorganism.

Aspect 33. The method of any of aspects 26-32, wherein the target nucleic acid is from selected from the group comprising SARS-CoV-2 virus, influenza virus, Zika virus, Dengue virus, Chikungunya virus, Ebola virus, hepatitis A virus, hepatitis B virus, hepatitis C virus, human immunodeficiency viruses (HIV), Mayaro virus, Escherichia coli (E. coli), enterococci, Salmonellae, Streptococcus pneumoniae, Staphylococcus aureus and Pseudomonas aeruginosa.

Aspect 34. The method of aspect 32, wherein the target nucleic acid from the first liquid sample is SARS-CoV-2 virus and wherein the target nucleic acid from the second liquid sample is influenza virus.

Aspect 35. The method of aspect 30, wherein analysis of the detected nucleic acid is performed in an analysis device, the analysis device comprising a battery, a heater, and a temperature control circuit.

Aspect 36. The method of aspect 35, wherein the analysis device further comprises an imaging apparatus, and wherein the detection of nucleic acid amplification and the analysis are performed in real time.

Aspect 37. The method of aspects 35 or 36, wherein the detection and analysis are performed at a point of care.

Aspect 38. The method of any of aspects 36 or 37, wherein the analysis provides viral load information.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present invention defined in the following claims, the scope of which are to be accorded the broadest interpretation so as to encompass modifications and equivalent structures.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

It should be noted that measurements, amounts, and other numerical data can be expressed herein in a range format. It is also understood that there are a number of values disclosed herein, and that each value is also herein disclosed as “approximately” that particular value in addition to the value itself. For example, if the value “10” is disclosed, then “approximately 10” is also disclosed. Similarly, when values are expressed as approximations, by use of the antecedent “approximately,” it will be understood that the particular value forms a further aspect. For example, if the value “approximately 10” is disclosed, then “10” is also disclosed.

As used herein, the terms “about,” “approximately,” “at or about,” and “substantially equal” can mean that the amount or value in question can be the exact value or a value that provides equivalent results or effects as recited in the claims or taught herein. That is, it is understood that amounts, sizes, measurements, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, measurement, parameter or other quantity or characteristic is “about,” “approximate,” “at or about,” or “substantially equal” whether or not expressly stated to be such. It is understood that where “about,” “approximately,” “at or about,” or “substantially equal” is used before a quantitative value, the parameter also includes the specific quantitative value itself, unless specifically stated otherwise.

Where a range is expressed, a further aspect includes from the one particular value and/or to the other particular value. Where a range of values is provided, it is understood that each intervening value, to the tenth of the unit of the lower limit unless the context dearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

1. A multiplex device for preparing a sample for nucleic acid detection comprising:
at least two parallel buffer units comprising a plurality of buffer wells arranged in a row, wherein each buffer well comprises a ball valve in a bottom of the well;
a mixing unit associated with each buffer unit, wherein the mixing unit comprises a mixing well and a pin, wherein the mixing unit is slidably connected to a bottom of the buffer unit such that the mixing unit can be moved along a length of the buffer unit to align with each buffer well in turn, wherein when the mixing unit is aligned with a buffer well the pin engages with the ball valve to release buffer from the buffer well into the mixing well; and
a detection unit removably coupled to the bottom of each mixing unit to receive fluids from the mixing well;
wherein the parallel buffer units are connected to form a multiplex device.

2. The device of claim 1, further comprising an amplification unit coupled to an end of each buffer unit, the amplification unit comprising a buffer well comprising a ball valve in the bottom of the well, wherein the mixing unit is slidably connected to a bottom of the amplification unit such that the mixing unit can be moved along a length of the amplification unit after the buffer unit to align with an amplification well, wherein when the mixing unit is aligned with the amplification well the pin engages with the ball valve to release buffer from the amplification well into the mixing well; and
wherein the amplification unit is selected from a LAMP unit for processing DNA or an RT-LAMP unit for processing RNA.

3. The device of claim 1, further comprising a drain unit removably coupled to the bottom of each detection unit to receive fluids from the detection unit.

4. The device of claim 1, wherein the detection unit comprises an absorbent layer, wherein the absorbent layer is selected from chromatography paper, cellulose paper, a membrane, and glass microfiber paper.

5. The device of claim 1, wherein the buffer units are modular and are removably connected to each other along a side of the buffer unit.

6. The device of claim 1, wherein the buffer units are permanently connected to each other.

7. The device of claim 1, wherein the device is comprised of 2 to 10 buffer units.

8. The device of claim 5, wherein a first well of each buffer unit can receive a sample, and wherein the sample can be the same for each buffer unit or not the same for each buffer unit.

9. The device of claim 3, wherein the drain unit comprises a tube, such that buffer can be extracted from the drain unit by a syringe or vacuum.

10. The device of claim 2, wherein each buffer unit comprises four buffer wells and each LAMP unit comprises a single well.

11. The device of claim 1, further comprising a heating unit, wherein the heating unit comprises a seat for the detection unit, wherein the heating unit provides a temperature of 57° C. to 67° C.

12. The device of claim 11, wherein the heating unit further comprises at least one cartridge heater.

13. The device of claim 11, wherein the heating unit is powered by a portable electronic device or batteries.

14. The device of claim 11, wherein the heating unit is a component of a portable analysis device, the analysis device further comprising a battery, a microscope, and a temperature control circuit, wherein the heating unit has a cap that provides a seat for the detection unit.

15. The device of claim 14, further comprising an imaging apparatus, wherein the imaging apparatus detects a signal of nucleic acid amplification in the detection unit in real time.

16. The device of claim 1, further comprising a sample collection unit, wherein the sample collection unit comprises a sample well having a ball valve in a bottom of the sample well, wherein when a first buffer well receives a bottom of the sample collection unit the ball valve of the collection unit is engaged to release contents of the sample collection unit into the buffer well.

17. A multiplex device for detecting nucleic acid from a nucleic acid source comprising:
at least two parallel buffer units, wherein each buffer unit comprises a plurality of buffer wells arranged in a row, wherein each buffer well comprises a ball valve in a bottom of the well;
wherein a nucleic acid source and a first buffer is provided to a first buffer well, a second buffer is provided to a second buffer well, a third buffer is provided to a third buffer well, and a fourth buffer is provided to a fourth buffer well;
an amplification unit coupled to an end of each buffer unit, the amplification unit comprising a buffer well, wherein the coupling forms a singleplex unit;
wherein if the nucleic acid source is DNA the amplification unit is a LAMP unit and if the nucleic acid source is RNA the amplification unit is an RT-LAMP unit;
wherein an amplification buffer is provided to an amplification buffer well;
a mixing unit associated with each singleplex unit, wherein the mixing unit comprises a mixing well and a pin, wherein the mixing unit is slidably connected to a bottom of the singleplex unit such that the mixing unit can be moved ag a length of the singleplex unit to align with each buffer well in turn beginning with the first buffer well and ending with the amplification unit, wherein when the mixing unit is aligned with a buffer well the pin engages with the ball valve to release buffer from the buffer well into the mixing well such that nucleic acid is produced in the mixing unit from the nucleic acid source after the mixing unit engages with the amplification unit;
a detection unit removably coupled to the bottom of each mixing unit to receive buffer and nucleic acid from the mixing well, wherein the nucleic acid is collected and amplified in the detection unit; and
a drain unit removably coupled to the bottom of each detection unit to extract buffer from the detection unit; and
a heating unit to amplify the nucleic acid product wherein the heating unit comprises a seat for the detection unit, wherein the heating unit provides a temperature of 57° C. to 67° C.

18. The device of claim 17, wherein the detection unit can be removed from the buffer unit and analyzed to determine whether liquid received by the detection unit contains nucleic acid from a target virus or microorganism.

19. The device of claim 18, wherein each detection unit can be configured to detect the same target virus or microorganism or a different target virus or microorganism.

20-25. (canceled)

26. A method for preparing a sample to be used for detecting nucleic acid from a nucleic acid source comprising:
placing a first liquid sample in at least a first well of a first buffer unit of a nucleic acid detecting apparatus;
placing at least a second, third and fourth liquid in at least second, third and fourth wells, respectively, of the first buffer unit of the nucleic acid detecting apparatus;
placing a second liquid sample in at least a first well of a second buffer unit of the nucleic acid detecting apparatus;
placing at least a second, third and fourth liquid in at least second, third and fourth wells, respectively, of the second buffer unit of the nucleic acid detecting apparatus;
wherein a mixing unit is slidably coupled to a bottom of each buffer unit allowing a user to create relative movement between the mixing unit and the buffer unit by exerting a manual force on at least one of the buffer unit and the mixing unit; and
exerting the manual force on at least one of the buffer unit and the mixing unit to slide the mixing unit along the buffer unit to cause the first, second, third and fourth wells to come into temporary alignment with the mixing unit for respective time periods;
wherein a valve mechanism in each of the wells causes the first, second, third and fourth liquids to be released from the first, second, third and fourth wells, respectively, into the mixing unit, in turn, when the manual force is exerted to cause the second, third and fourth wells to come into temporary alignment with the mixing unit for respective time periods such that each liquid sample is prepared for nucleic acid amplification as it combines with the buffers from the wells in the mixing unit; and
wherein a detection unit is coupled to a bottom of the mixing unit and receives fluid from the mixing unit.

27-38. (canceled)
